(19) United States
(12) Patent Application Publication
(10) Pub. No.: US 2026/0256887 A1
(43) Pub. Date: Sep. 3, 2026

(54) THERAPEUTICS FOR CANCER WITH ANTISENSE AND INTERFERON-GAMMA

(71) Applicant: GMP BIOTECHNOLOGY LIMITED, Hong Kong (CN)

(72) Inventor: Vuong TRIEU, Agoura Hills, CA (US)

(21) Appl. No.: 19/546,752

(22) Filed: Feb. 23, 2026

Related U.S. Application Data
(63) Continuation of application No. PCT/US2024/044867, filed on Aug. 30, 2024.
(60) Provisional application No. 63/535,431, filed on Aug. 30, 2023.
(60) Provisional application No. 63/567,406, filed on Mar. 19, 2024.

(51) Int. Cl. A61K 38/21; A61P 35/00; C12N 15/113

(52) U.S. Cl. CPC A61K 38/217; A61P 35/00; C12N 15/1136

(57) ABSTRACT

This invention relates to agents, compositions, and methods for use in treating or ameliorating symptoms of cancer. Exemplary synergistic therapies include the use of antisense oligonucleotide agents for suppressing expression of TGF-β2, both alone and in combination with interferon-gamma. One or more biomarkers can be used to select subjects for treatment.

### SEQUENCE LISTING

This application includes a sequence listing submitted electronically as an ST.26 file created on Aug. 27, 2024, named 018988-013WO1_SL.xml, which is 120,204 bytes in size and is hereby incorporated by reference in its entirety.

### TECHNICAL FIELD

This invention relates to agents, compositions, and methods for use in treating or ameliorating symptoms of cancer. Exemplary synergistic therapies include active agents for suppressing expression of TGF-β2, both alone and in combination with interferon-gamma. One or more biomarkers can be used to select subjects for treatment.

### BACKGROUND

Cancer is a complex pathology involving multiple variant cellular pathways. Because of this complexity, many anti-cancer drugs have limited or partial therapeutic effectiveness.

Drawbacks of conventional therapies include lack of efficacy as determined by overall survival. In some cases, surgical resection is not possible because of anatomical location of tumors.

Further drawbacks of conventional therapies include significant unwanted side effects such as killing healthy cells in addition to killing cancer cells.

Additional drawbacks of anti-cancer agents include high toxicity at required levels of therapeutic administration. In some cases, a monotherapy may not be a feasible strategy because the presence and level of therapeutic targets may vary.

What is needed are agents, compositions and methods for cancer diseases to increase efficacy and reduce toxicity and unwanted side effects. Therapy involving a combination of active agents can improve therapeutic effects. Moreover, the guidance of biomarkers can be a powerful tool for improving therapies.

Therapeutic compositions of different agents are needed to supply significant anti-tumor effects and cancer immunotherapeutic effects and which can improve efficacy, reduce side effects and reduce adverse health effects. There is a need for improved guidance for use of such compositions by using appropriate biomarkers to select synergistic effects of the agents and compositions.

### BRIEF SUMMARY

This invention provides agents, compositions, and methods for use in treating or ameliorating the symptoms of cancer. Synergistic pharmaceutical therapies of this invention include use of potent anti-tumor agents, alone or in combination with interferon-gamma. Cancer therapeutic modalities of this disclosure include selecting patients and cancers for therapy via biomarkers and disease variants to improve outcomes.

Embodiments of this invention contemplate suppressing high levels of TGF-β2 expression, which may be done in combination with interferon-gamma and selecting patients who based on certain levels of biomarkers.

Further embodiments of this invention include suppressing high levels of TGF-2 expression alone and selecting patients based on a biomarker.

In some embodiments, methods and therapeutic strategies of this invention can include increased guidance for successful patient outcomes using appropriate biomarkers to select synergistic effects of the compositions and agents.

In some embodiments, selecting cancer patients who have elevated IFNGR2 can improve clinical outcomes.

Embodiments of this invention include the following:

An agent for suppressing expression of TGF-β2 in combination with interferon-gamma for use in treating or ameliorating symptoms of cancer in subjects in need, wherein the subjects can be selected who have (a) one or both of JAK1 and STAT1 reduced and (b) TGF-β2 and IFNGR2 elevated.

A method for treating or ameliorating the symptoms of cancer in subjects in need, wherein the method may comprise:
selecting the subjects who may have (a) one or both of JAK1 and STAT1 reduced and (b) TGF-β2 and IFNGR2 elevated;
administering a composition comprising an agent for suppressing expression of TGF-β2; and
administering a composition comprising interferon-gamma.

A composition comprising an agent for suppressing expression of TGF-β2 and a pharmaceutically acceptable carrier for use in the preparation of a medicament or for treating or ameliorating symptoms of a cancer in a subject in combination with interferon-gamma, wherein the subjects can be selected who have (a) one or both of JAK1 and STAT1 reduced and (b) TGF-β2 and IFNGR2 elevated.

The agent, method, or composition above, wherein the cancer may be brain or spinal cancer, glioma, glioblastoma, diffuse intrinsic pontine glioma (DIPG), diffuse midline glioma (DMG), diffuse hemispheric glioma, or leptomeningeal or brain metastasis, or wherein cells of the cancer exhibit somatic mutations comprising H2-K27M, H3-K27M, or H3-G34 genomic variants.

The agent, method, or composition above, wherein the agent for suppressing expression of TGF-β2 and the interferon-gamma can be administered concurrently, simultaneously, sequentially, or separately in time.

The agent, method, or composition above, wherein the composition and agents may be administered by infusion or injection.

The agent, method, or composition above, wherein the one or both of JAK1 and STAT1 may have an mRNA level reduced below a median of a control group of subjects having the same cancer, and TGF-β2 and IFNGR2 elevated above a median of a control group of subjects having the same cancer.

The agent, method, or composition above, wherein the agent for suppressing expression of TGF-β2 can be selected from Table 1 or Table 2, and chemically-modified variants thereof, LNA variants thereof, gapmer variants thereof, and any combination or pooling thereof.

The agent, method, or composition above, wherein the agent for inhibiting or suppressing expression of TGF-β2 may be C*G*G*C*A*T*G*T*C*T*A*T*T*T*T*G*T*A SEQ ID NO:137 (OT-101) or CGGCATGTCTATTTTGTA SEQ ID NO:1.

The agent, method, or composition above, wherein the interferon-gamma can be human, recombinant interferon-gamma.

The agent, method, or composition above, wherein the agent or composition may comprise a carrier of sterile water for injection, saline, isotonic saline, or a combination thereof.

The agent, method, or composition above, wherein the agent or composition can be substantially free of excipients.

The agent, method, or composition above, wherein the composition can be stable for at least 14 days in carrier at 37° C.

The agent, method, or composition above, wherein the subject upon the administration or use may have a reduced TGF-β2 expression.

The agent, method, or composition above, wherein the administration or use can decrease mortality rate at month 6, 12, 18, 24, 30, or 36.

The agent, method, or composition above, wherein the administration or use may increase survival rate at month 6, 12, 18, 24, 30, or 36.

The agent, method, or composition above, wherein the administration or use of the composition can be combined with a standard of care treatment for cancer, wherein the standard of care treatment comprises chemotherapy or radiation therapy.

An agent for suppressing expression of TGF-β2 for use in treating or ameliorating symptoms of cancer in subjects in need, wherein the subjects can be selected who have (a) elevated IFNGR2 and (b) TGF-β2 elevated.

A method for treating or ameliorating symptoms of cancer in subjects in need, wherein the method may comprise:
selecting the subjects who have (a) elevated IFNGR2 and (b) TGF-β2 elevated; and
administering a composition comprising an agent for suppressing expression of TGF-β2.

A composition comprising an agent for suppressing expression of TGF-β2 and a pharmaceutically acceptable carrier for use in the preparation of a medicament or for treating or ameliorating symptoms of a cancer in a subject, wherein the subjects can be selected who have (a) elevated IFNGR2 and (b) TGF-β2 elevated.

The agent, method, or composition above, wherein the cancer can be brain or spinal cancer, glioma, glioblastoma, diffuse intrinsic pontine glioma (DIPG), diffuse midline glioma (DMG), diffuse hemispheric glioma, or leptomeningeal or brain metastasis, or wherein cells of the cancer exhibit somatic mutations comprising H2-K27M, H3-K27M, or H3-G34 genomic variants.

The agent, method, or composition above, wherein the composition or agent may be administered by infusion or injection.

The agent, method, or composition above, wherein the subjects may have elevated IFNGR2 as an mRNA level below a median of a control group of subjects having the same cancer and TGF-β2 elevated above a median of a control group of subjects having the same cancer.

The agent, method, or composition above, wherein the agent for suppressing expression of TGF-β2 can be selected from Table 1 or Table 2, and chemically-modified variants thereof, LNA variants thereof, gapmer variants thereof, and any combination or pooling thereof.

The method, agent or composition above, wherein the agent for inhibiting or suppressing expression of TGF-β2 can be C*G*G*C*A*T*G*T*C*T*A*T*T*T*T*G*T*A SEQ ID NO:137 (OT-101) or CGGCATGTCTATTTTGTA SEQ ID NO:1.

The agent, method, or composition above, wherein the composition may comprise a carrier of sterile water for injection, saline, isotonic saline, or a combination thereof.

The agent, method, or composition above, wherein the composition can be substantially free of excipients.

The agent, method, or composition above, wherein the composition may be stable for at least 14 days in carrier at 37° C.

The agent, method, or composition above, wherein the subject upon the administration may have a reduced TGF-β2 expression.

The agent, method, or composition above, wherein the administration can decrease mortality rate at month 6, 12, 18, 24, 30, or 36.

The agent, method, or composition above, wherein the administration may increase survival rate at month 6, 12, 18, 24, 30, or 36.

The agent, method, or composition above, wherein the administration of the composition can be combined with a standard of care treatment for cancer, wherein the standard of care treatment comprises chemotherapy or radiation therapy.

### BRIEF DESCRIPTION OF THE DRAWINGS

FIG. 1 shows upregulation of IFNGR2 mRNA expression in brain tumor samples. There was a significant augmentation in the IFNGR2 mRNA levels in DMG samples compared with that in the normal pons tissue (1.58-fold increase; p=5.5×10-4). JAK1 and STAT1 were reduced.

FIG. 2 shows a Kaplan-Meier overall survival chart obtained in a study of clinical outcomes in glioma patients. FIG. 2 shows that when TGF-β2 levels were high, indicating high disease activity, surprisingly improved overall survival was found for high levels of IFNGR2 (logrank P=0.012). This example shows that IFNGR2 biomarker guided surprisingly improved efficacy for outcomes in treating brain cancer.

FIG. 3 shows a Kaplan-Meier overall survival chart obtained in a study of clinical outcomes in glioma patients. FIG. 3 shows that when TGF-β2 levels were high, indicating high disease activity, surprisingly improved overall survival was found for higher levels of IFNGR2 (upper curve, logrank p<0.001). This example shows that IFNGR2 biomarker guided surprisingly improved efficacy for outcomes in treating brain cancer.

FIG. 4 shows Cox proportional hazards measurements obtained in a study of clinical outcomes in glioma patients. FIG. 4 shows that when allowing an interaction term between levels of TGF-β2 and IFNGR2 for clinical outcomes in glioma, that TGF-β2 was essentially independent of IFNGR2.

FIG. 5 shows a Kaplan-Meier overall survival chart obtained in a study of clinical outcomes in glioma patients. FIG. 5 shows significantly improved overall survival was found for suppressed low levels of TGF-β2.

FIG. 6 shows a Kaplan-Meier overall survival chart obtained in a study of clinical outcomes in glioma patients. FIG. 6 shows surprisingly improved overall survival was found for relatively higher levels of JAK1 (upper curve, logrank p<0.001). This example shows that JAK1 biomarker guided surprisingly improved efficacy for outcomes in treating brain cancer.

FIG. 7 shows a Kaplan-Meier overall survival chart obtained in a study of clinical outcomes in glioma patients. FIG. 7 shows that surprisingly improved overall survival was found for relatively higher levels of STAT1 (upper curve, logrank p<0.001). This example shows that STAT1 biomarker guided surprisingly improved efficacy for outcomes in treating brain cancer.

### DETAILED DESCRIPTION OF THE DISCLOSURE

This invention relates to agents, compositions, and methods for use in treating or ameliorating symptoms of cancer. Exemplary synergistic therapies include active agents for suppressing expression of TGF-β2, both alone and in combinations with interferon-gamma. One or more biomarkers can be used to select subjects for treatment.

In some embodiments, one or more biomarkers can be used to select subjects for the method, agent or use. The methods, compositions and agents can be used in combination with chemotherapy and standard-of-care therapies for the same cancer.

Embodiments of this invention contemplate suppressing high levels of TGF-β2 expression in combination with interferon-gamma and selecting patients who have low levels of certain biomarkers. High intra-tumor TGF-β2 levels of mRNA expression in cancer patients were strongly associated with poor survival, so that suppressing expression of TGF-β2 has been shown therapeutic. The therapeutic outcomes for suppressing high levels of TGF-β2 expression in combination with interferon-gamma and selecting patients who have certain levels of one or more biomarkers IFNGR2, JAK1, and STAT1 mRNA expression can be surprisingly increased as shown by overall survival rates.

Further embodiments of this invention include suppressing high levels of TGF-β2 expression in combination with selecting patients who have certain levels of a biomarker. High intra-tumor TGF-β2 levels of mRNA expression in cancer patients were strongly associated with poor survival, and suppressing expression of TGF-β2 has been shown therapeutic. The therapeutic outcomes for suppressing high levels of TGF-β2 expression in combination with selecting patients who have high levels of biomarker IFNGR2 mRNA expression can be surprisingly increased as shown by overall survival rates.

In some embodiments, selecting cancer patients who have high IFNGR2 can improve clinical outcomes. Suppressing high expression of TGF-β2 in patients who have high expression of IFNGR2 can improve clinical outcomes. In some aspects, subjects may be selected for treatment who have (a) elevated IFNGR2 and (b) TGF-β2 elevated.

In further embodiments, suppressing high levels of TGF-β2 in combination with administering IFN-γ for patients who have low levels of either JAK1 or STAT1 can improve survival outcomes. IFN-γ may activate interferon-gamma receptors in various immune cells and activate Janus kinases JAK1 and JAK2 and the signal transducer and activator of transcription STAT1 and STAT3 pathways. In certain embodiments, interferon-gamma may be delivered to the tumor microenvironment. IFN-γ can be administered for treatment through various routes and delivery systems known in the art. In some aspects, subjects may be selected for treatment who have (a) one or more of JAK1 and STAT1 reduced, and (b) TGF-β2 and IFNGR2 elevated.

In certain embodiments, cancers of this disclosure to be treated may include any of brain or spinal cancer, glioma, glioblastoma, diffuse intrinsic pontine glioma (DIPG), diffuse midline glioma (DMG), diffuse hemispheric glioma, or leptomeningeal or brain metastasis, or wherein cells of the cancer exhibit somatic mutations comprising H3-K27M, H2-K27M, or H3-G34 genomic variants.

In some embodiments, cancers of this disclosure to be treated may have cells which exhibit somatic mutations comprising H3-K27M, H2-K27M, or H3-G34 genomic variants.

In further embodiments, cancers of this disclosure to be treated may have cells which exhibit somatic mutations comprising H3-K27M genomic variants.

In certain embodiments, cancers of this disclosure to be treated may include diffuse midline gliomas with cells which exhibit somatic mutations comprising H3-K27M genomic variants.

In some embodiments, diagnoses, genomic variants or biomarker transcript levels can be obtained or measured by methods and/or facilities known and/or compiled in the art, including next-generation sequencing, RNA-Seq, whole exome sequencing, tissue biopsy, liquid biopsy, and radiological methods.

As used herein, the terms patient and subject are interchangeable. A subject in need of cancer therapy as described herein may be a human or animal subject.

As used herein, the terms TGF-β2, TGFB2, and TGF-β2 are synonymous.

As used herein, the term agent can refer to one or more active compounds, a combination of active compounds, or a composition containing one or more active compounds and a carrier, and/or a solvent, and/or any number of excipients. In some embodiments, the composition may be a pharmaceutical composition. In certain embodiments, the composition may be a pharmaceutical composition containing a therapeutically effective amount of one or more active compounds. Formulations of active agents can be determined by those skilled in the art. Some examples of excipients are given in Remington's Pharmaceutical Sciences, Mack Publishing Co., Easton, Pa. 1975, and Liberman, H. A. and Lachman, L., Eds., Pharmaceutical Dosage Forms, Marcel Decker, New York, N.Y., 1980. Methods for determining a therapeutically effective amount of a compound are known in the art.

### Anti-Cancer Methods and Compositions

This invention includes methods for treating or ameliorating the symptoms of cancer in subjects in need. The method may comprise selecting the subjects who have (a) one or both of JAK1 and STAT1 reduced, and (b) TGF-β2 and IFNGR2 elevated, administering a composition comprising an agent for suppressing expression of TGF-β2; and administering a composition comprising interferon-gamma. Improved overall survival for cancer patients can be achieved even when the tumor microenvironment may be immunologically cold.

This invention further includes methods for treating or ameliorating the symptoms of brain or spine cancer in subjects in need. The method may comprise selecting subjects who have TGF-β2 and IFNGR2 elevated, and administering a composition comprising an agent for suppressing expression of TGF-β2.

In some embodiments, a method of this disclosure for treating or ameliorating the symptoms of cancer may include a step or steps for selecting subjects for treatment who have elevated expression of TGF-β2 along with certain levels of guiding biomarkers. Improved overall survival for cancer patients can be achieved even when the tumor microenvironment may be immunologically cold.

In further embodiments, a method of this disclosure for treating or ameliorating the symptoms of cancer may include a step or steps for selecting subjects for treatment who have elevated expression of TGF-β2 and IFNGR2.

In additional aspects, agents used in combination can be administered concurrently, simultaneously, sequentially, or separately in time.

### Human TGF-β2-Specific Phosphorothioate Antisense Oligodeoxynucleotide

An antisense oligonucleotide (ASO) can be a single-stranded deoxyribonucleotide, which may be complementary to an mRNA target. The antisense therapy may downregulate a molecular target, which may be achieved by induction of RNase H endonuclease activity that cleaves the RNA-DNA heteroduplex with a significant reduction of the target gene translation. Other ASO mechanisms can include inhibition of 5′ cap formation, alteration of splicing process such as splice-switching, and steric hindrance of ribosomal activity.

Antisense therapeutic strategies can utilize single-stranded DNA oligonucleotides that inhibit protein production by mediating the catalytic degradation of a target mRNA, or by binding to sites on mRNA needed for translation. Antisense oligonucleotides can be designed to target the viral RNA genome or viral transcripts. Antisense oligonucleotides can provide an approach for identifying potential targets, and therefore represent potential therapeutics.

Antisense oligonucleotides can be small synthetic pieces of single-stranded DNA that may be 15-30 nucleotides in length. An ASO may specifically bind to a complementary DNA/RNA sequence by Watson-Crick hybridization and once bound to the target RNA, inhibit the translational processes either by inducing cleavage mechanisms or by inhibiting mRNA maturation. An ASO may selectively inhibit gene expression with specificity. Chemical modifications of DNA or RNA can be used to increase stability.

For example, modifications can be introduced in the phosphodiester bond, the sugar ring, and the backbone. ASO antiviral agents may block translational processes either by (i) ribonuclease H (RNAse H) or RNase P mediated cleavage of mRNA or (ii) by sterically (non-bonding) blocking enzymes that are involved in the target gene translation. Human TGF-β2-specific phosphorothioate antisense oligodeoxynucleotide (OT-101; AP 12009; Trabedersen), hereafter referred to as OT-101 or AP 12009, is intended to reduce the level of TGF-β2 protein in malignant gliomas, and thereby delay the progression of disease.

Antisense oligodeoxynucleotides are short strings of DNA that are designed to downregulate gene expression by interfering with the translation of a specific encoded protein at the mRNA level. OT-101 is a synthetic 18-mer phosphorothioate oligodeoxynucleotide (S-ODN) where all 3′-5′ linkages are modified to phosphorothioates. The molecular formula is C177H208N60Na17094P17S17 and the molecular weight 6,143 g/mol. OT-101 can be designed to be complementary to a specific sequence of human TGF-β2 mRNA following expression of the gene.

OT-101 can be supplied as a lyophilized powder in 50 mL glass vials in three different quantities. Each vial is identified by the name of the investigational product, trial number, dosing group, mode of application, quantity of OT-101 contained (in mg), total volume after dissolving (in mL) and resulting concentration (in μM), name of sponsor, name of manufacturer, batch number, vial number, storage temperature, and expiry date. The study medication can be provided in closed units, packaged separately for each concentration. The packages may contain the appropriate vial(s) and all necessary components of the application system (i.e., syringes, tube, and filter). OT-101 lyophilized powder can be dissolved in isotonic (0.9%) aqueous sodium chloride prior to use.

Examples of agents of this disclosure for inhibiting or suppressing expression of TGF-β2 include TGF-β2-specific antisense oligonucleotides given in SEQ ID NOs: 1-136 in Table 1.

TABLE 1
TGF-β2-specific antisense oligonucleotides

| SEQ ID | | |
|---|---|---|
| NO: | Ref. | ANTISENSE SEQUENCE |
| 1 | 1676-1693 | CGGCATGTCT ATTTTGTA (OT-101) |
| 2 | — | TAAAGTTCGT TTAGAGAACA G |
| 3 | — | AGCCCTGTAT ACGAC |
| 4 | — | GTAGGTAAAA ACCTAATAT |
| 5 | — | CGTTTAGAGA ACAGATCTAC |
| 6 | — | CATTGTAGAT GTCAAAAGCC |
| 7 | — | CTCCCTCATG GTGGCAGTTG A |
| 8 | — | GTTCGTTTAG AGAACAGATC |
| 9 | 178-195 | TTTGTTCCTG GATGACTC |
| 10 | 179-196 | GTTTGTTCCT GGATGACT |
| 11 | 180-197 | AGTTTGTTCC TGGATGAC |
| 12 | 181-198 | CAGTTTGTTC CTGGATGA |
| 13 | 182-199 | TCAGTTTGTT CCTGGATG |
| 14 | 183-200 | CTCAGTTTGT TCCTGGAT |
| 15 | 716-733 | TGTGTGTGTG TGCGTGTG |
| 16 | 717-734 | GTGTGTGTGT GTGCGTGT |
| 17 | 718-735 | TGTGTGTGTG TGTGCGTG |
| 18 | 719-736 | GTGTGTGTGT GTGTGCGT |
| 19 | 720-737 | TGTGTGTGTG TGTGTGCG |
| 20 | 721-738 | GTGTGTGTGT GTGTGTGC |
| 21 | 722-739 | TGTGTGTGTG TGTGTGTG |
| 22 | 723-740 | GTGTGTGTGT GTGTGTGT |
| 23 | 724-741 | TGTGTGTGTG TGTGTGTG |
| 24 | 725-742 | GTGTGTGTGT GTGTGTGT |
| 25 | 726-743 | TGTGTGTGTG TGTGTGTG |
| 26 | 727-744 | GTGTGTGTGT GTGTGTGT |
| 27 | 728-745 | TGTGTGTGTG TGTGTGTG |
| 28 | 729-746 | GTGTGTGTGT GTGTGTGT |
| 29 | 730-747 | CGTGTGTGTG TGTGTGTG |
| 30 | 731-748 | GCGTGTGTGT GTGTGTGT |
| 31 | 732-749 | TGCGTGTGTG TGTGTGTG |
| 32 | 733-750 | GTGCGTGTGT GTGTGTGT |
| 33 | 971-988 | AGTGGCGGAT CTGAACTC |
| 34 | 972-989 | GAGTGGCGGA TCTGAACT |
| 35 | 1430-1447 | GAGTGTGCTG CAGGTAGA |
| 36 | 1432-1449 | TCGAGTGTGC TGCAGGTA |
| 37 | 1433-1450 | ATCGAGTGTG CTGCAGGT |
| 38 | 1570-1587 | GTGCTGTTGT AGATGGAA |
| 39 | 1571-1588 | GGTGCTGTTG TAGATGGA |
| 40 | 1572-1589 | TGGTGCTGTT GTAGATGG |
| 41 | 1573-1590 | CTGGTGCTGT TGTAGATG |
| 42 | 1574-1591 | CCTGGTGCTG TTGTAGAT |
| 43 | 1575-1592 | CCCTGGTGCT GTTGTAGA |
| 44 | 1576-1593 | TCCCTGGTGC TGTTGTAG |
| 45 | 1577-1594 | GTCCCTGGTG CTGTTGTA |
| 46 | 1578-1595 | AGTCCCTGGT GCTGTTGT |
| 47 | 1895-1912 | CTGGGTTGGA GATGTTAA |
| 48 | 1896-1913 | GCTGGGTTGG AGATGTTA |
| 49 | 1897-1914 | CGCTGGGTTG GAGATGTT |
| 50 | 1900-1917 | TAGCGCTGGG TTGGAGAT |
| 51 | 1903-1920 | ATGTAGCGCT GGGTTGGA |
| 52 | 1945-1962 | AGCCATTCGC CTTCTGCT |
| 53 | 1994-2011 | GTCTTTATGG TGAAGCCA |
| 54 | 1995-2012 | TGTCTTTATG GTGAAGCC |
| 55 | 1996-2013 | CTGTCTTTAT GGTGAAGC |
| 56 | 1997-2014 | CCTGTCTTTA TGGTGAAG |
| 57 | 2000-2017 | GTTCCTGTCT TTATGGTG |
| 58 | 2001-2018 | GGTTCCTGTC TTTATGGT |
| 59 | 2002-2019 | AGGTTCCTGT CTTTATGG |
| 60 | 2003-2020 | CAGGTTCCTG TCTTTATG |
| 61 | 2004-2021 | CCAGGTTCCT GTCTTTAT |
| 62 | 2183-2200 | GGTCTTCCCA CTGTTTTT |
| 63 | 2194-2211 | AGGAGATGTG GGGTCTTC |
| 64 | 2195-2212 | CAGGAGATGT GGGGTCTT |
| 65 | 2241-2258 | GGTTGGTCTG TTGTGACT |
| 66 | 2242-2259 | CGGTTGGTCT GTTGTGAC |
| 67 | 2243-2260 | CCGGTTGGTC TGTTGTGA |
| 68 | 2522-2539 | GAGAATGGTT AGAGGTTC |
| 69 | 2525-2542 | GTAGAGAATG GTTAGAGG |
| 70 | 2542-2559 | GGTGTTTTGC CAATGTAG |
| 71 | 2543-2560 | GGGTGTTTTG CCAATGTA |
| 72 | 2544-2561 | TGGGTGTTTT GCCAATGT |
| 73 | 2666-2683 | CATCATCGTT GTCGTCGT |
| 74 | 2979-2996 | GAACGGTACG TACAGCAA |
| 75 | 2980-2997 | GGAACGGTAC GTACAGCA |
| 76 | 2981-2998 | AGGAACGGTA CGTACAGC |
| 77 | 2982-2999 | TAGGAACGGT ACGTACAG |
| 78 | 2983-3000 | ATAGGAACGG TACGTACA |
| 79 | 2984-3001 | GATAGGAACG GTACGTAC |
| 80 | 2985-3002 | GGATAGGAAC GGTACGTA |
| 81 | 2986-3003 | GGGATAGGAA CGGTACGT |
| 82 | 3029-3046 | GGGTGCCTAT TGCATAGC |
| 83 | 3030-3047 | AGGGTGCCTA TTGCATAG |
| 84 | 3031-3048 | AAGGGTGCCT ATTGCATA |
| 85 | 3032-3049 | GAAGGGTGCC TATTGCAT |
| 86 | 3033-3050 | GGAAGGGTGC CTATTGCA |
| 87 | 3035-3052 | TGGGAAGGGT GCCTATTG |
| 88 | 3036-3053 | ATGGGAAGGG TGCCTATT |
| 89 | 3037-3054 | AATGGGAAGG GTGCCTAT |
| 90 | 3038-3055 | GAATGGGAAG GGTGCCTA |
| 91 | 3039-3056 | AGAATGGGAA GGGTGCCT |
| 92 | 3040-3057 | AAGAATGGGA AGGGTGCC |
| 93 | 3041-3058 | TAAGAATGGG AAGGGTGC |
| 94 | 3042-3059 | GTAAGAATGG GAAGGGTG |
| 95 | 3043-3060 | AGTAAGAATG GGAAGGGT |
| 96 | 3044-3061 | GAGTAAGAAT GGGAAGGG |
| 97 | 3259-3276 | CAGACTTTCT CGGTCATA |
| 98 | 3260-3277 | GCAGACTTTC TCGGTCAT |
| 99 | 3261-3278 | TGCAGACTTT CTCGGTCA |
| 100 | 3262-3279 | ATGCAGACTT TCTCGGTC |
| 101 | 3263-3280 | AATGCAGACT TTCTCGGT |
| 102 | 3264-3281 | TAATGCAGAC TTTCTCGG |
| 103 | 4281-4298 | GACCTGGACT TTTTTCCC |
| 104 | 4282-4299 | TGACCTGGAC TTTTTTCC |
| 105 | 4283-4300 | CTGACCTGGA CTTTTTTC |
| 106 | 4284-4301 | GCTGACCTGG ACTTTTTT |
| 107 | 4467-4484 | CTGCAATGAT GTGGCAAA |
| 108 | 4468-4485 | TCTGCAATGA TGTGGCAA |
| 109 | 4469-4486 | TTCTGCAATG ATGTGGCA |
| 110 | 4470-4487 | CTTCTGCAAT GATGTGGC |
| 111 | 5062-5079 | GCTGCCCACT TGCATACT |
| 112 | 5569-5586 | GTTGGCAGAA CATAGAAC |
| 113 | 5570-5587 | CGTTGGCAGA ACATAGAA |
| 114 | 5571-5588 | GCGTTGGCAG AACATAGA |
| 115 | 5616-5633 | ATGGGGCTAC AGGGGATA |
| 116 | 5617-5634 | TATGGGGCTA CAGGGGAT |
| 117 | 5618-5635 | TTATGGGGCT ACAGGGGA |
| 118 | 5620-5637 | AGTTATGGGG CTACAGGG |
| 119 | 5621-5638 | AAGTTATGGG GCTACAGG |
| 120 | 5622-5639 | CAAGTTATGG GGCTACAG |
| 121 | 5623-5640 | CCAAGTTATG GGGCTACA |
| 122 | 5624-5641 | TCCAAGTTAT GGGGCTAC |
| 123 | 5625-5642 | ATCCAAGTTA TGGGGCTA |
| 124 | 5626-5643 | TATCCAAGTT ATGGGGCT |
| 125 | 5627-5644 | CTATCCAAGT TATGGGGC |
| 126 | 5759-5776 | ATTGGAGGAA ATAGGGTG |
| 127 | 5783-5800 | GTCTTGTAGG TAGCAGCC |
| 128 | 5784-5801 | GGTCTTGTAG GTAGCAGC |
| 129 | 5785-5802 | TGGTCTTGTA GGTAGCAG |
| 130 | 5786-5803 | CTGGTCTTGT AGGTAGCA |
| 131 | 5787-5804 | TCTGGTCTTG TAGGTAGC |
| 132 | 5788-5805 | GTCTGGTCTT GTAGGTAG |
| 133 | 5789-5806 | AGTCTGGTCT TGTAGGTA |
| 134 | 5790-5807 | GAGTCTGGTC TTGTAGGT |
| 135 | 5791-5808 | GGAGTCTGGT CTTGTAGG |
| 136 | 5792-5809 | AGGAGTCTGG TCTTGTAG |

The sequences of Table 1 can be chemically-modified to provide active variants thereof, LNA variants thereof, as well as gapmer variants thereof, as known in the art. The sequences of Table 1 can be used in any combination as active agents, such as pooling combinations.

It is understood that additional antisense oligonucleotides of this disclosure can be constructed based on the TGF-β2 gene sequence.

In some embodiments, an agent of antisense sequences can be gapmers formed by adding 1 to 5 protected ribo-nucleotides on each flank of the phosphorothioate deoxy-nucleotide sequences in Table 1. For example, the ribo-nucleotides can be protected with 2′-OMe, 2′-OEt, or 2′-O-MOE substituents, or with LNA, cMOE, or cEt bridges, as well as phosphorothioate linkages.

In some embodiments, an agent of antisense sequences can be a n-M-n RNA (2′-OMe)*-DNA*-RNA (2′-OMe)* gapmer, where n is from 3-7 and M is from 6-12. In certain embodiments, the gapmer can be a 3-10-3 or 5-10-5 LNA*-DNA*-LNA* or cEt*-DNA*-cEt* gapmer (* designates phosphorothioate linkages).

Examples of agents of this disclosure for inhibiting or suppressing expression of TGF-β2 include TGF-β2-specific phosphorothioate antisense oligonucleotides given in SEQ ID NOs: 137-144 in Table 2, based on the sequences in Table 1.

TABLE 2
TGF-B2-specific phosphorothioate antisense oligonucleotides

| SEQ ID | | |
|---|---|---|
| NO: | Ref. | ANTISENSE SEQUENCE |
| 137 | 1676-1693 | C*G*G*C*A*T*G*T*C*T*A*T*T*T*T*G*T*A (OT-101) |
| 138 | — | G*T*T*C*G*T*T*T*A*G*A*G*A*A*C*A*G*A*T*C |
| 139 | — | T*A*A*A*G*T*T*C*G*T*T*T*A*G*A*G*A*A*C*A*G |
| 140 | — | A*G*C*C*C*T*G*T*A*T*A*C*G*A*C |
| 141 | — | G*T*A*G*G*T*A*A*A*A*A*C*C*T*A*A*T*A*T |
| 142 | — | C*G*T*T*T*A*G*A*G*A*A*C*A*G*A*T*C*T*A*C |
| 143 | — | C*A*T*T*G*T*A*G*A*T*G*T*C*A*A*A*A*G*C*C |
| 144 | — | C*T*C*C*C*T*C*A*T*G*G*T*G*G*C*A*G*T*T*G*A |

Embodiments of this invention further include pharmaceutical compositions for inhibiting or suppressing expression of TGF-β, or for treating or ameliorating the symptoms of cancer in a human or animal. The pharmaceutical compositions may contain a TGF-β suppressing agent, pharmaceutically acceptable salts forms, esters, polymorphs or stereoisomers thereof, and any combination thereof, as well as a carrier. The TGF-β suppressing agent may be selected from TGF-β2-specific antisense oligonucleotides. The carrier may be sterile water for injection, saline, isotonic saline, or a combination thereof.

Importantly, a composition of this disclosure may be substantially free of excipients. Compositions of this invention which are substantially free of excipients have been found to be surprisingly stable in a carrier. In some embodiments, the composition may be stable for at least 14 days, or at least 21 days, or at least 28 days in a carrier at 37° C.

In additional embodiments, a pharmaceutical composition for infusion may contain less than 1% by weight of excipients, or less than 0.5% by weight of excipients, or less than 0.1% by weight of excipients.

### OT-101 Antisense Oligonucleotide

The API trabedersen/OT-101 is a synthetic 18-mer S-ODN consisting of the bases adenine (A), thymine (T), guanine (G), and cytosine (C), with all 3′-5′ linkages modified to phosphorothioates. The thioate modification can make the drug more resistant to degradation, resulting in an increased stability in vitro and in vivo. Its molecular structure (nucleotide sequence) can be designed to be complementary to a specific sequence of human transforming growth factor-beta 2 (TGF-β2) mRNA. This sequence can be selected among related molecules for its superior chemical and structural properties, biological activity, and specificity to achieve the best antisense effects in vitro and in vivo.

The chemical structure, exemplary of the phosphorothioate moieties (C-A-G), and the physical characteristics of trabedersen are shown in Table 3.

TABLE 3
Chemical and Physical Characteristics of Trabedersen
Chemical and Physical Characteristics of Trabedersen

| | | |
|---|---|---|
| 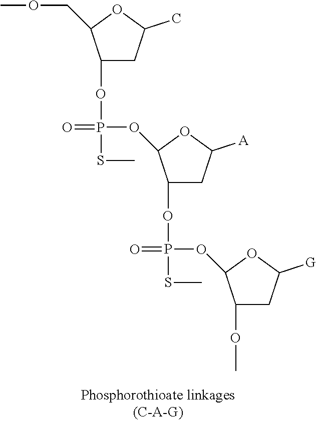 | Appearance: Molecular Formula (sodium salt, anhydrous): Molecular Weight (sodium salt, anhydrous): Number of moieties: Backbone characteristics: Polymorphism: Water content: Solubility: pH of solution: | White to off-white solid C₁₇₇N₂₀₈N₆₀O₉₄P₁₇S₁₇Na₁₇ 6143 g/mol 18 Phosphorothioates No crystalline morphology in the solid Up to 15% Very soluble in water (>50 mg/mL) Soluble in methanol (>8.5 mg/mL) Insoluble in iso-propanol (0.01 mg/mL) 6-8 |

The IMP can be supplied as a sterile lyophilizate for solution for infusion in 50H glass vials (primary container) containing 7.37 mg trabedersen (intratumoral treatment) and in 20R glass vials (primary container) containing 250 mg trabedersen (intravenous treatment), respectively. No excipients may be in the finished drug product. Glass vials are commonly used for parenterals. Sterile rubber stoppers appropriate for lyophilization can seal the glass vial. The stopper may be sealed with a crimping capsule that includes a colored flip-off cap. For clinical use, each vial can be provided within a white-colored folding box to protect the vials from light exposure and damage during transport. Both the glass vials and the folding boxes can be labeled according to local requirements. The primary and secondary containers of the closure system can fulfill international quality standards for the packaging of sterile solid drug products for injections.

### Brain and Spine Cancer Biomarkers

Biomarker mRNA levels of transforming growth factor beta 2 (TGF-β2) and interferon gamma receptor 2 (IFNGR2) can have biomarker potential in highly aggressive cancers. Expression levels of IFNGR2 and TGF-β2 can be upregulated in cancer compared with normal tissue.

In some embodiments, an agent for suppressing expression of TGF-β2 may be used in combination with interferon-gamma for use in treating or ameliorating symptoms of cancer in subjects in need, wherein the subjects are selected who have (a) one or both of JAK1 and STAT1 reduced and (b) TGF-β2 and IFNGR2 elevated.

In further embodiments, methods for treating or ameliorating the symptoms of cancer in subjects in need may include steps for selecting the subjects who have (a) one or both of JAK1 and STAT1 reduced and (b) TGF-β2 and IFNGR2 elevated; administering a composition comprising an agent for suppressing expression of TGF-β2; and administering a composition comprising interferon-gamma.

Embodiments of this invention also contemplate compositions comprising an agent for suppressing expression of TGF-2 and a pharmaceutically acceptable carrier for use in the preparation of a medicament or for treating or ameliorating symptoms of a cancer in a subject in combination with interferon-gamma, wherein the subjects are selected who have (a) one or both of JAK1 and STAT1 reduced and (b) TGF-β2 and IFNGR2 elevated.

Improved overall survival for cancer patients can be achieved even when the tumor microenvironment may be immunologically cold.

Multivariate Cox proportional hazards can be used to show that TGF-β2 can be a prognostic cancer indicator. Survival outcomes in cancer patients can indicate the abrogation of target mRNA expression, for example TGF-β2, in an immunologically cold tumor microenvironment can be used to treat cancer patients.

Furthermore, inclusion of interferon-gamma (IFN-γ) to stimulate and activate JAK1 and STAT1 in anti-tumor APC cells present the tumor microenvironment can enhance the effect of target mRNA expression blockade, for example blockade of TGF-β2.

For example, Diffuse Midline Gliomas (DMGs) are malignant primary glial tumors that occur in all age groups but are more prevalent in children and are responsible for 10-20% of all the central nervous system tumors and almost one-third of the high-grade gliomas in pediatric patients.

Understanding of DMGs has significantly grown because of improved biopsy techniques. For example, epigenetic investigations have identified H3K27 alterations common in DMGs and similar midline gliomas within the thalamus, spine, and brainstem regions. Alterations can include mutations in H3.3, H3.1, and H3.2, as well as EZHIP gene overexpression and EGFR mutants. One type of diffuse high-grade glioma is H3 K27 altered. Other types of gliomas include H3 wildtype, IDH (isocitrate dehydrogenase) wildtype, and infant-type gliomas.

Conventional treatment by surgical resection may not possible because of the tumor's anatomical location in the pons adjacent to cranial nerve nuclei involved with autonomic functions essential for life.

A standard of care management for DMG involves fractionated radiotherapy over a six-week course, with a total dose of 54 Gy. Cytotoxic chemotherapeutic drugs include temozolomide, gemcitabine, capecitabine, targeted agents such as tyrosine kinase inhibitors, and stem cell transplantation. However, in general, none have shown significantly improved overall survival rates. Additional therapies include adoptive immunotherapy toxicities, vaccines, oncolytic virus therapy, and immune checkpoint inhibitors.

Embodiments of this invention include utilizing therapeutic guidance from molecules in the brainstem.

In some embodiments, suppressing TGF-β2 can be used for tumor therapy, which can reduce immune suppression and immune evasion, as well as tumor progression. Suppressing TGF-β2 can release immunosuppression of tumors. Activating certain elements of tumor microenvironment with interferon-gamma (IFN-γ) can promote proinflammatory cytokine synthesis, enhanced phagocytosis, and increased tumor antigen-presenting capacity to improve the anti-tumor response.

In certain embodiments, suppressing TGF-β2 can be used in combination with administration of IFN-γ for tumor therapy and can provide enhanced cancer patient outcomes.

In further embodiments, TGF-β2 can be specific for overall and progression-free survival in brain cancer. Cancer patients who can be selected for treatment may exhibit high levels of tumor TGF-β2 mRNA in combination with elevated levels of mRNA coding for an IFN-γ receptor. Cancer patients who can be selected for treatment may exhibit high levels of tumor TGF-β2 mRNA in combination with reduced levels of mRNA coding for IFN-γ signaling molecules.

In additional embodiments, a therapy may be used for patients with low levels of downstream interferon signaling markers and high levels of TGF-β2 mRNA to improve survival rates. A therapy targeting elevated TGF-β2 levels can also provide enhanced anti-tumor response and overall survival.

In further embodiments, biomarkers which can be used include a level of a tumor mutation burden (TMB), a level of a tumor neoantigen, a level of a tumor-associated immune cell, or a combination thereof. In some embodiments, biomarkers which can be used are a level of a basophil cell, a B-cell, a T-cell, a T-helper cell (Th), an eosinophil cell, a macrophage cell, a mesenchymal stem cell, or a combination thereof. In certain embodiments, biomarkers which may be used include a level of a CD4+ cell, a memory T-cell, a CD8+ cell, a natural killer T-cell, a regulatory T-cell, a type 1 T-helper cell (Th1), a type 2 T-helper cell (Th2), or a combination thereof.

In some embodiments, one or more components of a tumor microenvironment can be determined by means known in the art, including analysis of fresh frozen and FFPE tissue samples by various techniques, microarray screening, immune cell and subtype analysis by various techniques, as well as genome stability, TMB, expression profiling, and various next generation sequencing (NGS) techniques.

### Active Agents

An antisense oligonucleotide may be supplied as a lyophilized powder in glass vials in different quantities. Antisense oligonucleotide lyophilized powder can be dissolved in isotonic (0.9%) aqueous sodium chloride prior to use.

In some examples and embodiments, an antisense oligonucleotide agent may be administered or used by infusion at a dose of 4 μl/min at a dose level of 10 μM on Days 1 to 7, or at a dose of 20 μM on Days 1 to 7, or at a dose of 40 μM on Days 1 to 7, or at a dose of 80 μM on Days 1 to 7.

In some examples and embodiments, an antisense oligonucleotide agent may be administered or used by infusion at a dose of 4 μl/min, or 2-8 μl/min, at a dose level of 2 μM on Days 1 to 7, or at a dose of 4 μM on Days 1 to 7, or at a dose of 8 μM on Days 1 to 7, or at a dose of 10 μM on Days 1 to 7.

In some embodiments, an antisense oligonucleotide agent may be administered by injection at concentrations of 61.43 mg/ml (10 μM), 1 mg/ml, 7.35 mg/ml, 15 mg/ml, or 18.23 mg/ml.

In further embodiments, an antisense oligonucleotide agent may be administered or used by infusion, either singly, in combination with a formulation-compatible drug, or in combination with standard of care therapies.

An agent of this disclosure may be a pharmaceutically-acceptable salt, salt polymorph, ester, or isomer, and one or more pharmaceutically acceptable excipients. Excipients may comprise any one or more pharmaceutically acceptable excipients selected from diluents, stabilizers, disintegrants and anticaking agents. In some embodiments, the excipients may comprise any one or more of microcrystalline cellulose, polysorbate 80, crospovidone, croscarmellose sodium, and magnesium stearate.

A pharmaceutical compositions may contain an active agent as well as a pharmaceutically-acceptable carrier. The carrier may be sterile water for injection, saline, isotonic saline, or a combination thereof.

Importantly, a composition of this disclosure may be substantially free of excipients. Compositions of this invention which are substantially free of excipients can be surprisingly stable in a carrier. In some embodiments, the composition may be stable for at least 14 days, or at least 21 days, or at least 28 days in a carrier at 37° C.

In additional embodiments, a pharmaceutical composition for infusion may contain less than 1% by weight of excipients, or less than 0.5% by weight of excipients, or less than 0.1% by weight of excipients.

Embodiments of this invention further contemplate therapeutic modalities in which a composition of this invention is administered or utilized in combination with a standard of care therapy for the disease.

In further embodiments, a therapeutically effective amount of an antisense agent can be from 0.1 to 3000 mg per day, or 1 to 1000 mg per day, or 2 to 500 mg per day, or 2 to 200 mg per day.

In certain embodiments, a formulation of an antisense agent can have a concentration of from 0.05 to 50 μM, or 0.1 to 25 μM, or 0.1 to 10 μM, or 0.1 to 7.5 μM, or 0.1 to 5 μM.

In certain embodiments, a method for using an antisense agent can use an effective dosage amount of from 1 to 1000 mg/m2/day, or from 1 to 500 mg/m2/day, or from 1 to 250 mg/m²/day, or from 1 to 100 mg/m²/day, or from 1 to 50 mg/m²/day. Mean human body surface area can be about 1.6 to 1.9 m².

In additional embodiments, a method for using an antisense agent can use an effective dosage amount of from 0.05 to 40 mg/kg/day, or from 0.1 to 30 mg/kg/day, or from 0.2 to 20 mg/m²/day, or from 0.3 to 10 mg/m²/day, or from 0.5 to 5 mg/m²/day. Mean human body weight can be about 60 kg.

In certain embodiments, agents of this disclosure may be prepared from a lyophilized powder of the agent.

In some examples and embodiments, an agent may be administered or used by injection or infusion at a dose of 4 μl/min at a dose level of 10 μM on the Days 1 to 7, or at a dose of 20 μM on Days 1 to 7, or at a dose of 40 μM on Days 1 to 7, or at a dose of 80 μM on Days 1 to 7.

In certain embodiments, an antisense oligonucleotide may be supplied as a sterile lyophilizate for solution prior to administration in 20R glass vials with a quantity of 250 mg/vial. The lyophilizate may be reconstituted aseptically in sterile, preservative-free isotonic NaCl solution. Antisense oligonucleotide solution can be administered every 14 days using a portable pump system as a continuous i.v. infusion on days 4-7 according to a 4-days-on, 10-days-off schedule. A schedule may be 7 d on/7d off and 4 d on/10 d off scheduling. A dose may be 40, 80, 160, 140, 190, 250, 330 mg.

In certain embodiments, an agent may be administered or used by injection or infusion at a dose of 40, 80, 160, 140, 190, 250, 330 mg/m² on Days 1 to 7, or at a dose of 40, 80, 160, 140, 190, 250, 330 mg/m² on Days 1 to 4.

In some examples and embodiments, an agent may be administered or used by injection or infusion at a dose of 4 μl/min, or 2-8 μl/min, at a dose level of 2 μM on Days 1 to 7, or at a dose of 4 μM on Days 1 to 7, or at a dose of 8 μM on Days 1 to 7, or at a dose of 10 μM on Days 1 to 7.

As used herein, the term chemically-modified can refer to LNA variants and gapmer variants. Antisense agents of this disclosure can be used by pooling in a formulation, or used in any combination.

### Methods and Compositions for Cancer

The agents of this invention can be used for treating cancer or ameliorating the symptoms of cancer in a human subject or animal in need. The agents may be prepared in a pharmaceutical composition for injection or infusion.

A pharmaceutical composition for injection or infusion may be administered in a therapeutically sufficient amount to the subject.

Methods and compositions for administering interferon-gamma are known in the art. Human IFN-γ may be recombinantly produced and may be carrier-free. Interferon-gamma is a dimerized soluble cytokine that is the only member of the type II class of interferon. IFN-gamma has been used in a wide variety of clinical indications. Interferon-gamma is a regulator of the immune response and signals via the Janus Activated Kinase (JAK)-Signal Transducer and Activator of Transcription (STAT) pathways. IFN-γ is the major interferon produced by mitogenically or antigenically stimulated lymphocytes. See Entrez Gene ID 3458 (Human).

Pharmaceutical agents or active substances of this disclosure may be dissolved or suspended in a physiological solvent or in any other appropriate solvent. The agents may be in the form of a free base, or a salt, hydrate, ester, amide, enantiomer, isomer, tautomer, polymorph, prodrug, or derivative of these compounds. The above mentioned agents as well as combinations thereof can be used in the apparatuses, methods, kits, combinations, and compositions herein described.

Compositions of this invention can contain compounds to be infused to the subject formulated as an injectable formulation, for example, an aqueous solution or suspension of the compounds suitable for intravenous delivery. When preparing the composition for injection, particularly for intravenous delivery, illustratively, the continuous phase comprises an aqueous solution of tonicity modifiers, buffered to a pH below 7.4, for example, or below 7 or below 6.6, for example. The tonicity modifiers comprise, for example, sodium chloride, glucose, mannitol, trehalose, glycerol, or other pharmaceutical agents that renders the osmotic pressure of the formulation isotonic with blood.

The system of this invention can contain a preservative added to the formulation. A preservative includes benzalkonium chloride, propylparaben, butylparaben, chlorobutanol, belizyl alcohol, phenol, sodium benzoate, or EDTA.

The composition of this disclosure can contain a pharmaceutically acceptable carrier. The carrier materials that can be employed in making the compositions of the present invention are any of those commonly used excipients in pharmaceutics and should be selected on the basis of compatibility with the pharmaceutical agent and the release pro-file properties of the desired dosage form.

A composition of this invention can contain excipients such as are given in Remington's Pharmaceutical Sciences, Mack Publishing Co., Easton, Pa. 1975, and Liberman, H. A. and Lachman, L., Eds., Pharmaceutical Dosage Forms, Marcel Decker, New York, N. Y., 1980.

Numbered embodiments of this invention include the following:
(1) An agent for suppressing expression of TGF-β2 in combination with interferon-gamma for use in treating or ameliorating symptoms of cancer in subjects in need, wherein the subjects are selected who have (a) one or both of JAK1 and STAT1 reduced and (b) TGF-β2 and IFNGR2 elevated.
(2) A method for treating or ameliorating the symptoms of cancer in subjects in need, the method comprising:
selecting the subjects who have (a) one or both of JAK1 and STAT1 reduced and (b) TGF-β2 and IFNGR2 elevated;
administering a composition comprising an agent for suppressing expression of TGF-β2; and
administering a composition comprising interferon-gamma.
(3) A composition comprising an agent for suppressing expression of TGF-β2 and a pharmaceutically acceptable carrier for use in the preparation of a medicament or for treating or ameliorating symptoms of a cancer in a subject in combination with interferon-gamma, wherein the subjects are selected who have (a) one or both of JAK1 and STAT1 reduced and (b) TGF-β2 and IFNGR2 elevated.
(4) The agent, method, or composition of any of embodiments 1-3, wherein the cancer is brain or spinal cancer, glioma, glioblastoma, diffuse intrinsic pontine glioma (DIPG), diffuse midline glioma (DMG), diffuse hemispheric glioma, or leptomeningeal or brain metastasis, or wherein cells of the cancer exhibit somatic mutations comprising H2-K27M, H3-K27M, or H3-G34 genomic variants.
(5) The agent, method, or composition of any of embodiments 1—, wherein the agent for suppressing expression of TGF-β2 and the interferon-gamma are administered concurrently, simultaneously, sequentially, or separately in time.
(6) The agent, method, or composition of any of embodiments 1-5, wherein the composition and agents are administered by infusion or injection.
(7) The agent, method, or composition of any of embodiments 1-6, wherein the one or both of JAK1 and STAT1 have an mRNA level reduced below a median of a control group of subjects having the same cancer, and TGF-β2 and IFNGR2 elevated above a median of a control group of subjects having the same cancer.
(8) The agent, method, or composition of any of embodiments 1-7, wherein the agent for suppressing expression of TGF-β2 is selected from Table 1 or Table 2, and chemically-modified variants thereof, LNA variants thereof, gapmer variants thereof, and any combination or pooling thereof.
(9) The agent, method, or composition of any of embodiments 1-8, wherein the agent for inhibiting or suppressing expression of TGF-β2 is

SEQ ID NO: 137
C*G*G*C*A*T*G*T*C*T*A*T*T*T*T*G*T*A
(OT-101)
or
SEQ ID NO: 1
CGGCATGTCTATTTTGTA.

(10) The agent, method, or composition of any of embodiments 1-9, wherein the interferon-gamma is human, recombinant interferon-gamma.
(11) The agent, method, or composition of any of embodiments 1-10, wherein the agent or composition comprises a carrier of sterile water for injection, saline, isotonic saline, or a combination thereof.
(12) The agent, method, or composition of any of embodiments 1-11, wherein the agent or composition is substantially free of excipients.
(13) The agent, method, or composition of any of embodiments 1-12, wherein the composition is stable for at least 14 days in carrier at 37° C.
(14) The agent, method, or composition of any of embodiments 1-13, wherein the subject upon the administration or use has a reduced TGF-β2 expression.
(15) The agent, method, or composition of any of embodiments 1-14, wherein the administration or use decreases mortality rate at month 6, 12, 18, 24, 30, or 36.
(16) The agent, method, or composition of any of embodiments 1-15, wherein the administration or use increases survival rate at month 6, 12, 18, 24, 30, or 36.
(17) The agent, method, or composition of any of embodiments 1-16, wherein the administration or use of the composition is combined with a standard of care treatment for cancer, wherein the standard of care treatment comprises chemotherapy or radiation therapy.
(18) An agent for suppressing expression of TGF-β2 for use in treating or ameliorating symptoms of cancer in subjects in need, wherein the subjects are selected who have (a) elevated IFNGR2 and (b) TGF-β2 elevated.
(19) A method for treating or ameliorating symptoms of cancer in subjects in need, the method comprising:
selecting the subjects who have (a) elevated IFNGR2 and (b) TGF-β2 elevated; and
administering a composition comprising an agent for suppressing expression of TGF-β2.
(20) A composition comprising an agent for suppressing expression of TGF-β2 and a pharmaceutically acceptable carrier for use in the preparation of a medicament or for treating or ameliorating symptoms of a cancer in a subject, wherein the subjects are selected who have (a) elevated IFNGR2 and (b) TGF-β2 elevated.
(21) The agent, method, or composition of any of embodiments 18-20, wherein the cancer is brain or spinal cancer, glioma, glioblastoma, diffuse intrinsic pontine glioma (DIPG), diffuse midline glioma (DMG), diffuse hemispheric glioma, or leptomeningeal or brain metastasis, or wherein cells of the cancer exhibit somatic mutations comprising H2-K27M, H3-K27M, or H3-G34 genomic variants.
(22) The agent, method, or composition of any of embodiments 18-21, wherein the composition or agent is administered by infusion or injection.
(23) The agent, method, or composition of any of embodiments 18-22, wherein the subjects have elevated IFNGR2 as an mRNA level below a median of a control group of subjects having the same cancer and TGF-β2 elevated above a median of a control group of subjects having the same cancer.
(24) The agent, method, or composition of any of embodiments 18-23, wherein the agent for suppressing expression of TGF-β2 is selected from Table 1 or Table 2, and chemically-modified variants thereof, LNA variants thereof, gapmer variants thereof, and any combination or pooling thereof.
(25) The method, agent or composition of any of embodiments 18-24, wherein the agent for inhibiting or suppressing expression of TGF-β2 is

SEQ ID NO: 137
C*G*G*C*A*T*G*T*C*T*A*T*T*T*T*G*T*A
(OT-101)
or
SEQ ID NO: 1
CGGCATGTCTATTTTGTA.

(26) The agent, method, or composition of any of embodiments 18-25, wherein the composition comprises a carrier of sterile water for injection, saline, isotonic saline, or a combination thereof.
(27) The agent, method, or composition of any of embodiments 18-26, wherein the composition is substantially free of excipients.
(28) The agent, method, or composition of any of embodiments 18-27, wherein the composition is stable for at least 14 days in carrier at 37° C.
(29) The agent, method, or composition of any of embodiments 18-28, wherein the subject upon the administration has a reduced TGF-β2 expression.
(30) The agent, method, or composition of any of embodiments 18-29, wherein the administration decreases mortality rate at month 6, 12, 18, 24, 30, or 36.
(31) The agent, method, or composition of any of embodiments 18-30, wherein the administration increases survival rate at month 6, 12, 18, 24, 30, or 36.
(32) The agent, method, or composition of any of embodiments 18-31, wherein the administration of the composition is combined with a standard of care treatment for cancer, wherein the standard of care treatment comprises chemotherapy or radiation therapy.

All publications including patents, patent application publications, and non-patent publications referred to in this description, as well as the sequence listing are each expressly incorporated herein by reference in their entirety for all purposes.

Although the foregoing disclosure has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation. This invention includes all such additional embodiments, equivalents, and modifications. This invention includes any combinations or mixtures of the features, materials, elements, or limitations of the various illustrative components, examples, and claimed embodiments.

The designations of agents, compounds and structures of this disclosure are meant to encompass all possible isomers, stereoisomers, diastereomers, enantiomers, and/or optical isomers that would be understood to exist for the specified structure, including any mixture, racemic or otherwise, thereof.

### EXAMPLES

Example 1. This example shows IFNGR2 mRNA is surprisingly elevated in brain cancer.

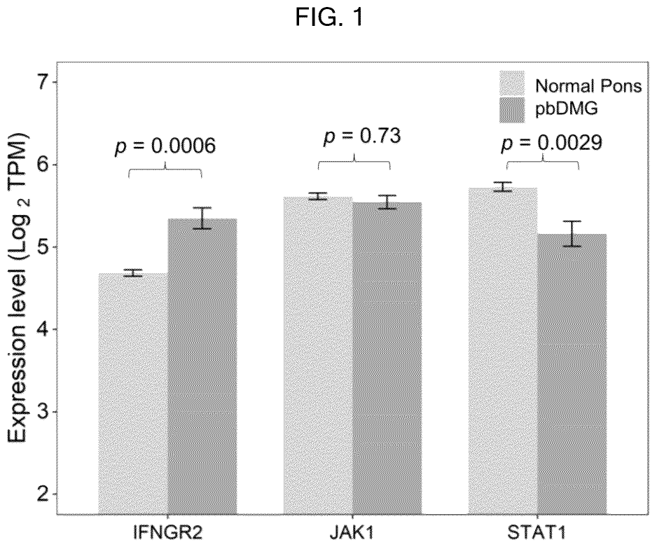

FIG. 1 shows upregulation of IFNGR2 mRNA expression in pbDMG tumor samples. IFNGR2, JAK1, and STAT1 mRNA expression levels (log 2-transformed TPM) for primary tumor samples for pbDMG patients were detected, including molecular subtype classifications of DMG, H3K27M (n=23); DMG, H3K27M and TP53 (n=8); HGG, H3 wildtype (n=2); HGG, H3 wildtype and TP53 (n=1); HGG, to be classified (n=10) and 1 not determined, for brain tumors localized to the pons/brainstem. IFNGR2 (n=45), JAK1 (n=45), and STAT1 (n=45) mRNA expression levels (log 2 TPM) in DMG samples were compared with the expression levels in normal pons samples from 29 pons regions The expression for these 29 normal pons regions was determined by averaging the TPM values for 2-8 independent samples/regions from 21 subjects. The bar charts illustrate mean expression levels for mRNA in tumor specimens from DMG patients (dark gray bars) compared with those in normal pons samples (light gray bars). The statistical significance of differences in mRNA expression levels (in log 2-transformed TPM values) was assessed using a two-way ANOVA with linear contrasts and FDR-adjusted p-values.

There was a significant augmentation (increase) in the IFNGR2 mRNA levels in DMG samples compared with that in the normal pons tissue (1.58-fold increase; p=5.5× 10-4).

There was some reduction in JAK1 and significant reduction in STAT1 consistent with dysregulation of the IFN signaling pathway.

FIG. 1 shows significant upregulation of IFNGR2 mRNA levels compared with those in normal pons tissue (1.58-fold increase; p=5.5×10−4) was observed in pbDMG patients. Examination of the STAT1 expression showed significantly lower levels of expression (p=0.0029) in pbDMG tumors compared with those in normal brainstem/pons tissue. These clinical facts evidence the role of these biomarkers in pbDMG tumors and dysregulation of IFN signaling.

Example 2. This example shows that administering exogenous IFN-γ to IFN-γ-starved DMG tumors achieves increased STAT1 and JAK1. The combination of exogenous IFN-γ with suppressing TGFB2, achieved with antisense such as OT-101, achieves surprisingly improved efficacy for outcomes in treating brain cancer.

In a study of clinical outcomes for pediatric DMG patients, IFN-γ was not expressed in three-quarters of the patients, and its maximum expression was less than 1 part per million transcripts, with an upper quartile expression of 0.64 TPM. This was a very low level of expression. Despite the relative absence of IFN-γ, its receptor IFNGR2 was surprisingly upregulated.

Selecting cancer patients who have TGF-β2 high and IFNGR2 surprisingly upregulated and treating with a combination of exogenous IFN-γ and suppressing TGF-β2 achieves surprisingly improved efficacy for outcomes.

Without wishing to be bound by theory, exogenous IFN-γ treatment achieves increased expression of downstream STAT1 and JAK1. Thus, administering exogenous IFN-γ to IFN-γ-starved DMG tumors achieves improved outcomes.

Selecting cancer patients who have TGF-β2 high, IFNGR2 surprisingly upregulated, and either JAK1 or STAT1 low and treating with a combination of exogenous IFN-γ and suppressing TGF-β2 achieves surprisingly improved efficacy for outcomes.

| ## | | n | events | median | 0.95LCL | 0.95UCL |
|---|---|---|---|---|---|---|
| ## | TGFB2_Gene2 = a_Low.TGFB2_a_Low.IFNG | 14 | 13 | 10 | 7 | NA |
| ## | TGFB2_Gene2 = a_Low.TGFB2_High.IFNG | 6 | 6 | 13 | 10 | NA |
| ## | TGFB2_Gene2 = High.TGFB2_a_Low.IFNG | 15 | 15 | 8 | 6 | 17 |
| ## | TGFB2_Gene2 = High.TGFB2_High.IFNG | 7 | 7 | 11 | 3 | NA |

### Univariate Cox for IFN-γ (Upper Quartile for High Expression):

HR was determined for the High.IFNG group of patients (HR (95% CI range)=1.04 (0.53-2.04); P=0.909).

Multivariate Cox for TGF-β2 (Median cut-off) and IFNG (Upper quartile cut-off):

HR was determined for High. TGF-β2 group of patients (HR (95% CI range)=1.04 (0.46-2.34); P=0.933).

HR was determined for High. IFNG group of patients (HR (95% CI range)=0.94 (0.35-2.52); P=0.907).

HR was determined for age as a covariate (HR (95% CI range)=1.02 (0.95-1.08); P=0.624)

HR was determined for the interaction term (HR (95% CI range)=1.2 (0.31-4.67); P=0.793).

Example 3. This example shows that IFNGR2 biomarker guided surprisingly improved efficacy for outcomes in treating brain cancer.

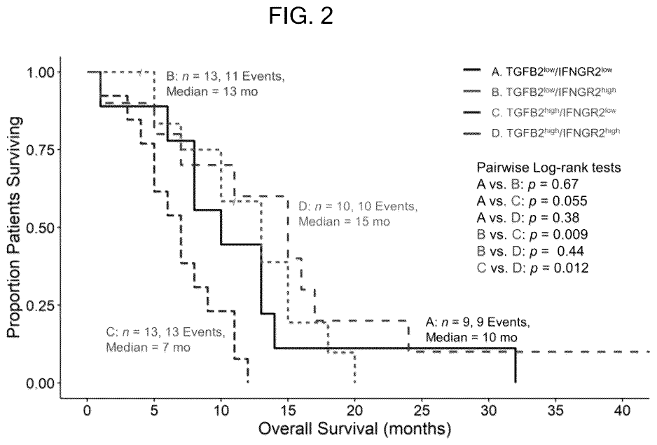

FIG. 2 shows a Kaplan-Meier overall survival chart obtained in a study of clinical outcomes in glioma patients. FIG. 2 shows that when TGF-β2 levels were high, indicating high disease activity, surprisingly improved overall survival was found for high levels of IFNGR2 (logrank P=0.012).

FIG. 2 shows that pbDMG patients with high levels of TGF-β2 and low levels of IFNGR2 mRNA expression exhibited significantly shorter overall survival (OS) times than patients with high levels of IFNGR2 mRNA expression. RNA-sequencing-based mRNA expression data was analyzed for 45 patients diagnosed with pbDMG (cBioPortal) The RSEM-determined TPM metric was used to calculate the percentiles of TGF-β2 and IFNGR2 expressions in 45 pbDMG patients. Four patient groups were then formed based on their expression levels of TGF-β2 and IFNGR2:
(A) low expressions of both, TGFB2low/IFNGR2low (lower than those of both TGF-β2 and IFNGR2 in the 50th percentile);
(B) combinations of high and low expression levels for both, TGFB2low/IFNGR2high;
(C) TGFB2high/IFNGR2low, and
(D) high expression levels of both, TGFB2high/IFNGR2high, higher than or equal to those of both TGF-β2 and IFNGR2 in the 50th percentile.

OS curves were then compared between these groups to assess the survival impacts of the combinations of TGF-β2 and IFNGR2 levels. The patient groups' survival times were recorded as follows:
TGFB2low/IFNGR2low had a median survival time of 10 months (with 95% CI ranging from 8 to NA and 9 events);
TGFB2low/IFNGR2high had a median survival time of 13 months (with 95% CI ranging from 10 to NA and 11 events);
TGFB2high/IFNGR2low had a median survival time of 7 months (with 95% CI ranging from 5 to NA and 13 events); and
TGFB2high/IFNGR2high had a median survival time of 15 months (with 95% CI ranging from 7 to NA and 10 events).

Examination of pairwise log-rank differences in OS times showed significant differences for the comparison between the TGFB2low/IFNGR2high and TGFB2high/IFNGR2low groups of patients (p=0.009) and for the comparison between the TGFB2high/IFNGR2low and TGFB2high/IFNGR2high groups of patients (p=0.012).

Suppressing TGF-β2 expression for pbDMG patients with increased IFNGR2 expression was associated with improved OS outcomes. Selecting DMG cancer patients who have increased IFNGR2 expression and suppressing TGF-β2 expression achieves surprisingly improved OS outcomes.

Impacts of TGF-β2 and IFNGR2 expression levels on the overall survival of four groups of pbDMG patients were studied. Low levels of mRNA expression were compared for both TGF-β2 and IFNGR2 (TGFB2low/IFNGR2low; lower than those of both TGF-β2 and IFNGR2 in the 50th percentile), combinations of high and low expression levels for TGF-β2 and IFNGR2 (TGFB2low/IFNGR2high and TGFB2high/IFNGR2low), and high expressions of both TGF-β2 and IFNGR2 (TGFB2high/IFNGR2high; higher than or equal to those of both TGF-β2 and IFNGR2 the 50th percentile).

Examination of pairwise differences for median OS times showed significant differences for the comparison between the TGFB2low/IFNGR2high (median=13 months (95% CI=10-NA months)) and TGFB2high/IFNGR2low groups of patients (median=7 months (95% CI=5-NA months); p=0.009) and for the comparison between the TGFB2high/IFNGR2low and TGFB2high/IFNGR2high (median=15 months (95% CI=7-NA months)) groups of patients (p=0.012). These results suggest that high levels of IFNGR2 confer a significant survival benefit at high TGF-β2 mRNA expression levels.

However, pbDMG patients with low levels of IFNGR2 and high levels of either TGFB1 or TGFB3 did not exhibit worse OS times, suggesting that the prognostic impact of TGF-β2 in the context of low IFNGR2 expression was surprising and specific to TGFB2.

Example 4. This example shows that IFNGR2 biomarker guided surprisingly improved efficacy for outcomes in treating brain cancer.

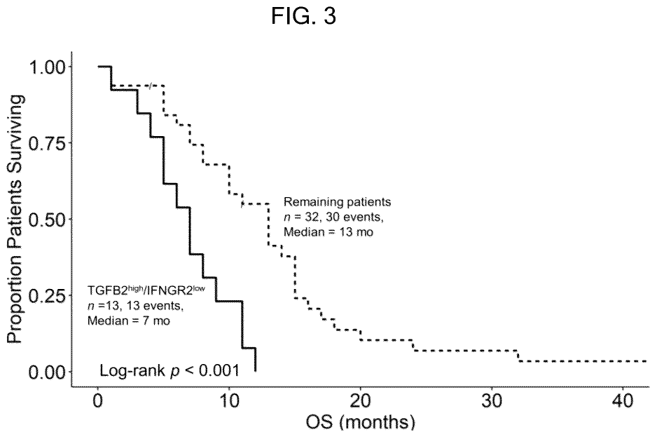

FIG. 3 shows a Kaplan-Meier overall survival chart obtained in a study of clinical outcomes in glioma patients. FIG. 3 shows that improved overall survival was found for relatively higher levels of IFNGR2 (upper curve, logrank p<0.001). In addition, suppressing high TGF-β2 levels, which indicated high disease activity, achieves surprisingly improved OS (upper curve). Selecting patients having relatively higher levels of IFNGR2 and suppressing high TGF-β2 levels achieves surprisingly improved overall survival, nearly doubled in median time. Because these effects were essentially independent, the improved survival outcomes indicated unexpected synergy by achieving improved outcomes that would not be achieved by either feature alone.

FIG. 3 shows that of the 45 patients who were studied, a group of 13 had high levels of TGF-β2 and low levels of IFNGR2 (lower curve). Their median overall survival time was 7 months, which significantly contrasted with that of the remaining group of 32 patients, with a median overall survival time of 13 months (95% CI=10-15 months). The survival outcome showed a significant difference between the two groups, with a log-rank chi-square value of 13.5 and a p-value of 2.3×10−4, thereby suggesting a significant prognostic impact on pbDMG patients.

Clinical metadata and RNA-sequencing-based mRNA expression data for 45 patients diagnosed with pbDMG were analyzed (cBioPortal). The RSEM-determined TPM metric was used to calculate the percentiles of TGF-β2 and IFNGR2 expressions in the 45 pbDMG patients. Two patient groups were then formed based on their expression levels of TGF-β2 and IFNGR2:
high expression of TGF-β2 mRNA and low expression of IFNGR2 (TGFB2high/IFNGR2low; higher than or equal to that of TGF-β2 in the 50th percentile and lower than that of IFNGR2 in the 50th percentile (n=13)); and
the “remaining patients” (n=32; pooled patients from the TGFB2low/IFNGR2high, TGFB2low/IFNGR2low, and TGFB2high/IFNGR2high groups).

Overall survival (OS) curves were then compared between these groups to assess the survival impact of the combination of the TGF-β2 and IFNGR2 levels. In the TGFB2high/IFNGR2low subset of patients, 8 were classified as DMG/H3K27M, 1 as DMG/H3K27M/TP53, 1 as HGG/H3 wildtype gene/IDH wildtype gene/TP53 mutation, 2 as HGG/to be classified, and 1 as NA according to the glioma grade and mutational status. In the remaining subset of patients, 15 were classified as DMG/H3K27M, 7 as DMG/H3K27M/TP53, 2 as HGG/H3 wildtype/IDH wildtype genes, and 8 as HGG/to be classified according to the glioma grade and mutational status.

The mean (±SEM) and median (range) log 2-TPM mRNA expression values of IFNGR2 in the TGFB2high/IFNGR2low subset of patients were 4.7±0.1 and 4.9 (3.7-5.4), respectively. For the “remaining patients” subset, the corresponding values were 5.6±0.1 and 5.7 (3.2-6.9).

The mean (±SEM) and median (range) log 2-TPM mRNA expression values of TGF-β2 in the TGFB2high/IFNGR2low subset of patients were 5.3±0.3 and 5.2 (4.1-7), respectively. For the “remaining patients” subset, the corresponding values were 3.5±0.3 and 3.3 (0.6-5.7).

In the group of 13 patients with TGFB2high/IFNGR2low, the median overall survival time was 7 months (95% CI=5-NA months; 13 events). In contrast, the median overall survival time for the 32 patients in the remaining group was 13 months. The 95% CI was between 10 and 15, with 30 events. Thus, a statistically significant difference in the survival outcome was observed between the two groups, with a log-rank chi-square value of 13.5 and a p-value of 2.3×10−4.

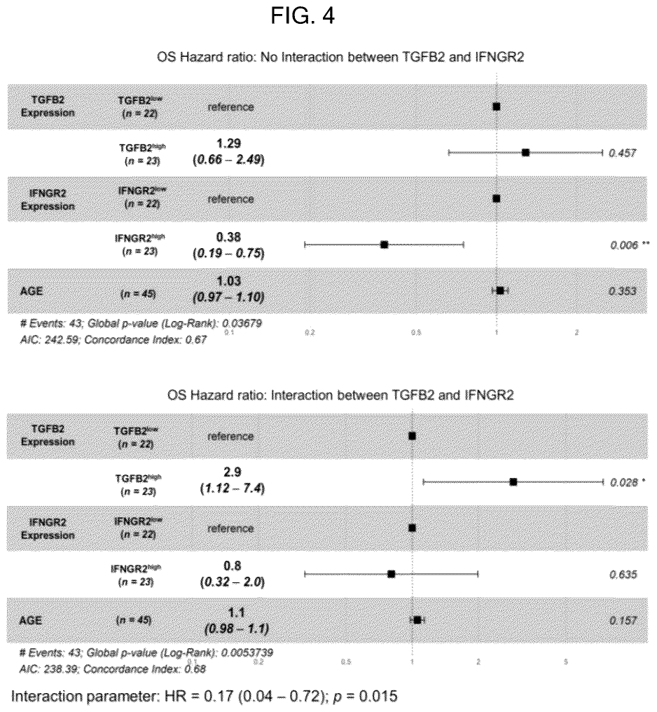

Example 5. FIG. 4 shows Cox proportional hazards measurements obtained in a study of clinical outcomes in glioma patients. FIG. 4 shows that when allowing an interaction term between levels of TGF-β2 and IFNGR2 for clinical outcomes in glioma, that TGF-β2 was essentially independent of IFNGR2. Because they were independent, the improved survival outcomes above indicated unexpected synergy by achieving improved outcomes that would not be achieved by either feature alone.

FIG. 4 shows pbDMG patients with high levels of TGF-β2 mRNA expression exhibited significantly increased hazard ratios in a multivariate Cox proportional hazards model considering age and the interaction between TGF-β2 and IFNGR2. Multivariate analyses of the potential effects of TGF-β2 and IFNGR2 levels on OS were determined using the multivariate Cox proportional hazards model to adjust for age by comparing models without and with TGF-β2 and IFNGR2 interactions. Clinical metadata and mRNA expression data for 45 patients diagnosed with pbDMG was analyzed (cBioPortal).

Both models included (i) the mRNA expression level for TGF-β2 as a categorical variable comparing high versus low TGF-β2 mRNA expression levels (50% cutoff for the range of TPM values); (ii) the mRNA expression level for IFNGR2 as a categorical variable comparing high versus low IFNGR2 mRNA expression levels (50% cutoff for the range of TPM values); and (iii) age as a linear covariate. Forest plots were utilized to visualize the hazard ratios for Cox proportional hazards models for OS outcomes.

The impact of including an interaction term as the fourth parameter in the Cox proportional hazards model (TGF-β2×IFNGR2) was analyzed to compare the independent effects of TGF-β2 and IFNGR2 in models with and without the interaction term.

The results of the model without an interaction term for the hazard ratios (HRs) showed that there was no significant increase in HR for the TGFB2high group of patients (HR (95% CI range)=1.29 (0.66-2.49); p=0.457). However, there was a significant decrease in HR for the IFNGR2high group of patients (HR (95% CI range)=0.38 (0.19-0.75); p=0.006). Additionally, there was no significant increase in HR for age as a linear covariate (HR (95% CI range)=1.03 (0.97-1.1); p=0.353).

The model that examined the interaction between IFNGR2 and TGF-β2 uncovered a significant increase in the hazard ratio for patients in the TGFB2high group (HR (95% CI range)=2.88 (1.12-7.39); p=0.028). However, there was no significant decrease in HR for patients in the IFNGR2high group (HR (95% CI range)=0.8 (0.32-1.99); p=0.635) or for age as a linear covariate (HR (95% CI range)=1.06 (0.98-1.14); p=0.157). The results also showed the significant effect of the interaction term (HR (95% CI range)=0.17 (0.04-0.72); p=0.015).

Overall, it was observed that the reduction in HR for the IFNGR2high group of patients was independent of TGF-β2 levels, and the inclusion of the interaction term in the model showed an increase in HR in the TGFB2high group of patients and was independent of IFNGR2 levels and the interaction between TGF-β2 and IFNGR2. The effect of IFNGR2 was captured in the interaction term.

In FIG. 4, * denotes p<0.05, ** denotes p<0.01.

Amplified TGF-β2 levels were an independent negative prognostic indicator for OS when controlling for age and IFNGR2 levels.

The effects of TGF-β2 and IFNGR2 levels in a multivariate context were investigated, including age as a linear control variable, utilizing a Cox proportional hazards models. This model, without an interaction term, showed that there was no significant increase in HR for the TGFB2high group of patients (HR (95% CI range)=1.29 (0.66-2.49); p=0.457). However, there was a significant decrease in HR for the IFNGR2high group of patients (HR (95% CI range)=0.38 (0.19-0.75); p=0.006), showing that high IFNGR2 levels have an overall pro-survival benefit.

The inclusion of an interaction term in the Cox proportional hazards model generated a more complex effect that uncovered a significant increase in the hazard ratio for patients in the TGFB2high group (HR (95% CI range)=2.88 (1.12-7.39); p=0.028), which was independent of IFNGR2 levels and age, and the significant pro-survival effect of high levels of IFNGR2 observed in the model without the interaction term was now captured in the significant effect of the IFNGR2×TGF-β2 interaction term ((HR (95% CI range)=0.17 (0.04-0.72); p=0.015). The parameters were analyzed in the Cox proportional hazards regression model, which factored in the interaction term for combinations of high and low TGF-β2 mRNA expression groups within the context of low levels and high levels of IFNGR2 mRNA expression in groups of patients. Patients in the TGFB2low group had a median OS time of 11 months, which was higher than the upper 95% confidence limit for the TGFB2high group (upper 95% confidence interval=10 months).

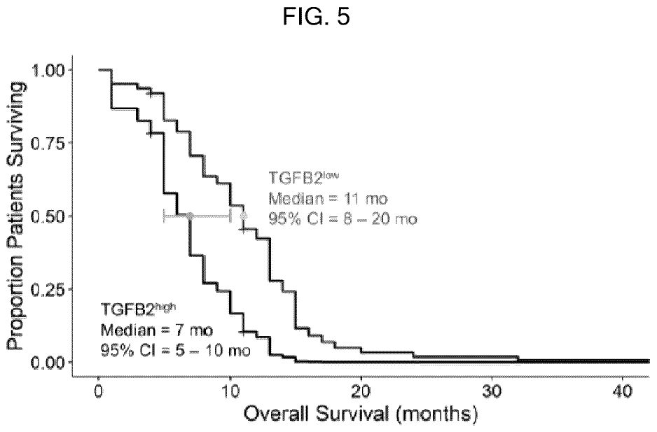

Example 6. This example shows that suppressing expression of TGF-β2 achieves surprisingly improved efficacy for outcomes in treating brain cancer. FIG. 5 shows a Kaplan-Meier overall survival chart obtained in a study of clinical outcomes in glioma patients.

FIG. 5 shows pbDMG patients with suppressed low levels of TGF-β2 mRNA expression exhibited significantly longer OS times. A multivariate Cox proportional hazards model was used considering age and the interaction between TGF-β2 and IFNGR2 levels. The survival proportion was calculated from the parameters in the Cox proportional hazards regression model, which included the interaction term for combinations of TGF-β2 high and low mRNA expression groups in the context of IFNGR2low mRNA expression group. Clinical metadata and mRNA expression data for 45 patients diagnosed with pbDMG was analyzed (cBioPortal).

The survival curves plot the shift in the OS curve for 45 pbDMG patients by comparing the median OS times for TGFB2high versus TGFB2low groups of patients in patients who expressed low levels of IFNGR2.

In the context of the low expression of IFNGR2, low levels of TGF-β2 resulted in more favorable OS times, whereby the median OS time of 11 months for the TGFB2low group of patients was greater than that of the upper 95% confidence limit for the TGFB2high group of patients (upper 95% confidence interval=10 months).

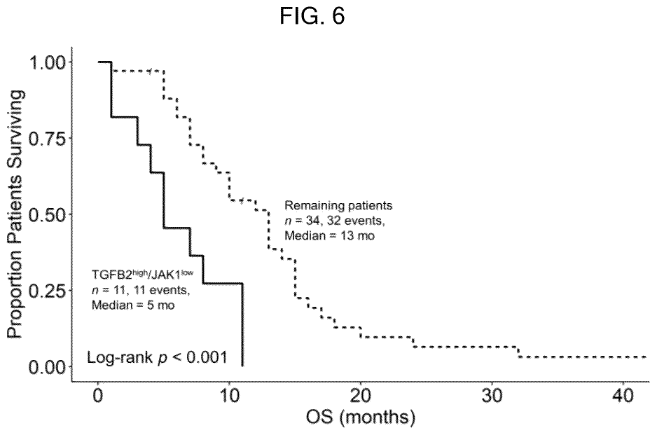

Example 7. This example shows that JAK1 biomarker guided surprisingly improved efficacy for outcomes in treating brain cancer by suppressing expression of TGFB2. FIG. 6 shows a Kaplan-Meier overall survival chart obtained in a study of clinical outcomes in glioma patients. FIG. 6 shows that increased levels of JAK1 surprisingly improved overall survival (upper curve, logrank p<0.001), as found in the “remaining patients” group.

In addition, suppressing high TGF-β2 levels, which indicated high disease activity, achieves surprisingly improved OS (upper curve). Selecting patients having relatively higher levels of JAK1 and suppressing high TGF-β2 levels achieves surprisingly improved overall survival, nearly tripled in median time. Because these effects were essentially independent, the improved survival outcomes indicated unexpected synergy by achieving improved outcomes that would not be achieved by either feature alone.

FIG. 6 shows pbDMG patients with relatively lower levels of TGF-β2 and relatively higher levels of JAK1 mRNA expression exhibited significantly longer OS times for the “remaining patients.” Clinical metadata and RNA-sequencing-based mRNA expression data for 45 patients diagnosed with pbDMG was analyzed (cBioPortal). The RSEM-determined TPM metric was used to calculate the percentiles of TGF-β2 and JAK1 expressions in the 45 pbDMG patients. Two patient groups were then formed based on their expression levels of TGF-β2 and JAK1:

high expression of TGF-β2 mRNA and low expression of JAK1 (TGFB2high/JAK1low; higher than or equal to that of TGF-β2 in the 50th percentile and lower than that of JAK1 in the 50th percentile (n=11)); and the remaining patients (n=34; pooled patients from the TGFB2low/JAK1high, TGFB2low/JAK1low, and TGFB2high/JAK1high groups).

In the TGFB2high/JAK1low and “remaining” subsets of patients, 8 and 15 were respectively classified as DMG/H3K27M; 1 and 7 were respectively classified as DMG/H3K27M/TP53; and 2 and 8 were respectively classified as HGG/to be classified. Additionally, in the remaining group of patients, 2 were classified as possessing HGG/H3 wildtype/IDH wildtype genes, 1 as possessing HGG/H3 wildtype/IDH wildtype/TP53 mutation, and 1 was classified as NA according to the glioma grade and mutational status.

The mean (±SEM) and median (range) log 2-TPM mRNA expression values for JAK1 in the TGFB2high/JAK1low subset of patients were 5.3±0.1 and 5.4 (4.7-5.5), respectively. For the “remaining” subset, the mean (±SEM) and median (range) were 5.6±0.1 and 5.7 (3.8-6.5), respectively.

The mean (±SEM) and median (range) log 2-TPM mRNA expression values for TGF-β2 in the TGFB2high/JAK1low subset of patients were 5.1±0.2 and 5.2 (4.2-6.7), respectively. For the “remaining” subset, the mean (±SEM) and median (range) were 3.6±0.3 and 3.4 (0.6-7), respectively.

Overall survival (OS) curves were then compared between these groups to assess the survival impact of the combination of the TGF-β2 and JAK1 levels. The median OS time of 11 patients in the TGFB2high/JAK1low group was 5 months (95% CI: 4-NA; number of events=11), while the median OS time of the 34 patients in the remaining group was 13 months (95% CI: 9-15; number of events=32). This difference in survival outcome between the two groups was surprisingly significant (log-rank chi-square value=13.5; p-value=2.4×10−4).

The impacts of JAK1 and STAT1 mRNA expression levels were investigated, which are downstream signaling molecules of IFNGR2 activation, in combination with TGF-β2 to assess the biochemical functional significance of the interaction observed between TGF-β2 and IFNGR2. OS curves were then compared between two groups of patients, TGFB2high/JAK1low versus the remaining patients, to assess the survival impact of the combination of the TGF-β2 and JAK1 levels.

FIG. 6 shows of the 45 patients who were studied, the 11 patients in the TGFB2high/JAK1low group had a median overall survival time of 5 months (95% CI: 4-NA; number of events=11), while the remaining 34 patients had a median overall survival time of 13 months (95% CI: 9-15; number of events=32). This difference in survival was found to be statistically significant (log-rank chi-square value=13.5; p-value=2.4×10−4). Similarly, the 11 patients in the TGFB2high/STAT1low group had a median overall survival time of 7 months (95% CI: 4-NA; number of events=11), while the remaining 34 patients had a median overall survival time of 13 months (95% CI: 8-15; number of events=32).

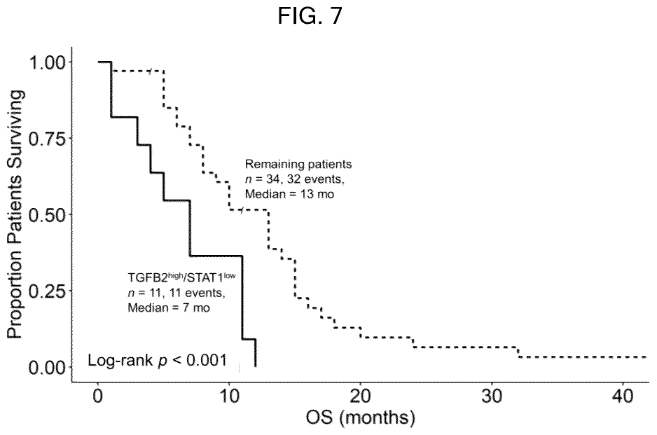

Example 8. This example shows that STAT1 biomarker guided surprisingly improved efficacy for outcomes in treating brain cancer. FIG. 7 shows a Kaplan-Meier overall survival chart obtained in a study of clinical outcomes in glioma patients. FIG. 7 shows that improved overall survival was found for relatively higher levels of STAT1 (upper curve, logrank p<0.001), as mainly found in the “remaining patients” group.

FIG. 7 shows pbDMG patients with relatively lower levels of TGF-β2 and relatively higher levels of STAT1 mRNA expression exhibited significantly longer OS times for the “remaining patients” group, nearly doubled in median time. Clinical metadata and RNA-sequencing-based mRNA expression data for 45 patients diagnosed with pbDMG was analyzed (cBioPortal). The RSEM-determined TPM metric was used to calculate the percentiles of TGF-β2 and STAT1 expressions in the 45 pbDMG patients. Two patient groups were then formed based on their expression levels of TGF-β2 and STAT1:
high expression of TGF-β2 mRNA and low expression of STAT1 (TGFB2high/STAT1low; higher than or equal to that of TGF-β2 in the 50th percentile and lower than that of STAT1 in the 50th percentile (n=11)); and
the remaining patients (n=34; pooled patients from the TGFB2low/STAT1high, TGFB2low/STAT1low, and TGFB2high/STAT1high groups).

In the TGFB2high/STAT1 low subset of patients, 6 were classified as possessing DMG with the H3K27M mutation, 1 as possessing DMG with the H3K27M and TP53 mutations, 1 as possessing HGG harboring H3 wildtype and IDH wildtype genes, 2 as possessing HGG with no mutational classification, and 1 as NA. In the remaining subset of patients, 17 were classified as possessing DMG with the H3K27M mutation, 7 as possessing DMG with the H3K27M and TP53 mutations, 2 as possessing HGG harboring H3 wildtype and IDH wildtype genes, and 8 as possessing HGG with no mutational classification.

The mean and median log 2-TPM mRNA expression values for STAT1 in the TGFB2high/STAT1low subset of patients were 4.3±0.2 and 4.4 (3.4-5.1), respectively. These values were 5.4±0.2 and 5.4 (3.1-8) for the “remaining” subset of patients.

The mean and median log 2-TPM mRNA expression values for the TGFB2high/STAT1low subset of patients were 5±0.2 and 5 (4.1-6.7), respectively. For the “remaining” subset of patients, these values were 3.7±0.3 and 3.4 (0.6-7), respectively.

Overall survival OS curves were then compared between these groups to assess the survival impact of the combination of the TGF-β2 and STAT1 levels.

The median OS time for 11 patients in the TGFB2high/STAT1low group was 7 months (95% CI: 4-NA; number of events=11), while the median OS time for the 34 patients in the remaining group was 13 months (95% CI: 8-15; number of events=32). The difference in the survival outcome between these groups was surprisingly significant (log-rank chi-square value=10.3; p-value=0.0014).

Example 9. Measurements of this disclosure include comparing mRNA expression levels in brain cancer tissue with normal brain tissue from the same anatomical location.

Transforming growth factor receptor ligands TGFB1, TGFB2, and TGFB3; IFN-γ receptor and downstream signaling molecules IFNGR2, JAK1, and STAT1; and mRNA transcript expression levels from RNA-seq experiments for brain tissues were acquired from the Human Protein Atlas version 23.0. Human tissues were anatomically dissected and analyzed using transcriptomics and mRNA samples from normal tissues extracted from frozen tissue sections. Following the sequencing, alignment, and quantification of the extracted nuclear RNA, the genes were annotated using database Ensembl version 109. TPM (transcripts) expression values were compiled from only the pons regions of the brain by filtering “Tissue Group” annotations in the accompanying description file. This data file included average levels of gene expression in 29 pons regions filtered using the keyword, “pons”, which retrieved values from the following regions: “anterior cochlear nucleus, ventral”; “dorsal cochlear nucleus”; “dorsal tegmental nucleus”; “dorsolateral tegmental area”; “Kolliker-Fuse nucleus”; “lateral lemniscus nuclei”; “lateral parabrachial nucleus”; “lateral vestibular nucleus”; “locus coeruleus”; “medial olivary nucleus”; “medial parabrachial nucleus”; “medial periolivary nuclei”; “motor facial nucleus”; “motor trigeminal nucleus”; “nuclei of the trapezoid body”; “paramedian reticular nucleus”; “pontine nuclei”; “pontine raphe nucleus”; “posteroventral cochlear nucleus”; “principal sensory trigeminal nucleus”; “reticular pontine nucleus, caudal”; “reticular pontine nucleus, oral”; “reticulotegmental nucleus”; “spinal trigeminal nucleus, oral”; “subcoeruleus area”; “superior olive”; “superior vestibular nucleus”; “ventral periolivary nuclei”; and “ventrolateral tegmental area, A5 NE cell group.”

mRNA expression levels of genes from Childhood Cancer Genomics (cBioPortal) were obtained and compared with mRNA expression levels in pbDMG tumors, using reported RNAseq TPM values. Data arrays for the mRNA expression values for each gene were normalized to “transcripts per million” (TPMs) for gene abundance values calculated using an RSEM alignment algorithm. Data was compiled using Open Pediatric Brain Tumor Atlas (OpenPBTA) and Pediatric Brain Tumor Atlas (PBTA, provisional) consortiums.

TPM expression values from the 29 pons regions of the brain were filtered with annotations under “tissue group” in the description file to compare with those of 45 pbDMG patients by applying a two-way ANOVA model to identify differentially expressed genes. The log 2-transformed TPM values for the genes (TGFB1, TGFB2, TGFB3, JAK1, STAT1, and IFNGR2) and tissues (29 normal pons tissues; 45 brainstem/pons specimens from pbDMG patients) were included as fixed factors, along with one interaction term to investigate gene-level effects for normal and pbDMG tissues (gene×tissue). For each gene, we conducted a comparison between normal pons and pbDMG samples and then determined the significance by adjusting the p-value using the false discovery rate algorithm provided in the R-package (FDR corrected for all the pairs in model 1 and blocked the design at the gene level in model 2). The calculations were performed in R using the multcomp_1.4-17 and emmeans 1.7.0 packages run in R, version 4.1.2, with the RStudio front end (RStudio 2021.09.0±351 “Ghost Orchid” Release). Bar chart graphics were constructed using the ggplot2_3.3.5 R package.

Example 10. Experimental comparison of characteristics for pbDMG patients with low-grade gliomas and high-grade gliomas and stratification of patient subsets relative to mRNA expression levels was made.

The clinical and RNA sequencing data for 45 pbDMG patients were analyzed to stratify patients according to mRNA expression levels. The diagnosis of pbDMG was primarily ascertained using radiological methods, which revealed borderless, diffuse, expansile, hyperintense lesions in the pons, which extended to other areas of the brainstem for all 45 patients. Forty-four of these patients had specified lesions from the pons, and one patient harbored the DMG/H3K27M mutation in the brainstem/medulla region.

Upon examining the clinical data file, it was reported that 31 of the 45 patients had diffuse midline gliomas with the H3K27M mutation (DMG/H3K27M; “diffuse midline glioma H3 K27 altered” according to the WHO 2021 classification scheme, which designates these tumors as Grade 4). Thirteen tumors were designated as WHO Grade 3/4 high-grade gliomas (HGGs), and one tumor had no designation (NA). Two HGG patients were reported to harbor both H3 and IDH wildtype genes, and one patient harbored H3 wildtype and IDH wildtype genes and a TP53 mutation.

Assays for mRNA expression values were obtained from 22 deceased patients: 1 at diagnosis, 20 from the initial CNS tumors, and 2 from progressive disease patients. The patient characteristics for these 45 pbDMG patients were compared using 171 pediatric high-grade gliomas and 404 low-grade gliomas obtained from the PBTA database (cBioPortal) and compiled using the Open Pediatric Brain Tumor Atlas (OpenPBTA) and Pediatric Brain Tumor Atlas (PBTA, provisional) consortiums.

The distribution of the age (median=7 years; (range=2-18 years)), fraction of the altered genome (median=0.16; (range=0-0.81)), and mutation count (median=23; (range=2-499)) for the 45 pbDMG patients were within the distribution observed for the HGG and LGG patients. The effect of the gene-level mRNA expression on OS outcomes was determined from patient-level data. The current regimens for treating pbDMG patients have established strategies involving the use of standard radiation therapy followed by specialized therapy with targeted FDA-approved drugs. The treatment is guided by gene expression analysis, whole-exome sequencing, and biomarkers. The TPM metric was used to calculate the percentiles of TGFB2, JAK1, STAT1, and IFNGR2 expression in 45 pbDMG patients. Four patient groups were then formed based on their expression levels of TGF-β2 and IFNGR2: high expressions of both (TGFB2high/IFNGR2high; higher than or equal to those of both TGF-β2 and IFNGR2 in the 50th percentile); low expressions of both (TGFB2low/IFNGR2low; lower than those of both TGF-β2 and IFNGR2 in the 50th percentile); and combinations of high and low expression levels for both (TGFB2high/IFNGR2low and TGFB2low/IFNGR2high.

Example 11. Experimental comparison of Overall Survival (OS) outcomes of pbDMG patients stratified relative to TGF-β2 and IFNGR2/JAK1/STAT1 mRNA expression levels was made.

OS curves were compared between groups to assess the survival impacts of the combinations of the four stratified groups of TGF-β2 and IFNGR2 levels. The impacts of TGFB2high/IFNGR2low, TGFB2high/JAK1low, and TGFB2high/STAT1low versus the remaining patients on the OS were determined to analyze the effect of the IFNGR2/JAK1/STAT1 axis on the survival of these patients. Comparisons of OS outcomes in the patient subsets were carried out using the Kaplan-Meier (KM) method, and the statistical significance was tested using the log-rank chi-square test and the following R-based software packages: survival_3.2-13, survminer_0.4.9, and survMisc_0.5.5. Graphical representations of the treatment outcomes were visualized using the following graph-drawing packages implemented in R: dplyr_1.0.7, ggplot2_3.3.5, and ggthemes_4.2.4. Considered were p-values less than 0.05 as significant after correcting for multiple comparisons across the four groups (6 comparisons), using the Benjamini-Hochberg method.

Example 12. Multivariate Analysis of OS Outcomes for pbDMG patients stratified relative to TGF-β2 and IFNGR2 mRNA expression levels and controlled for age and interaction of TGF-β2 and IFNGR2 was made.

To determine the impacts of the TGF-β2 and IFNGR2 levels on the OS, multivariate analyses were conducted using the Cox proportional hazards model, whereby age and the interaction between TGF-β2 and IFNGR2 were controlled for in the analysis. Briefly, the model included (i) the mRNA expression level for TGF-β2 as a categorical variable comparing high versus low TGF-β2 mRNA expression levels at a 50% cutoff for expression values; (ii) the mRNA expression level for IFNGR2 as a categorical variable comparing high versus low IFNGR2 mRNA expression levels (50% cutoff), and (iii) the age implemented in R (survival_3.2-13 run in R, version 4.1.2). Forest plots were utilized to visualize the hazard ratios for the Cox proportional hazards models for OS outcomes (survminer_0.4.9 run in R, version 4.1.2 (1 Nov. 2021)). The life table hazard ratios (HRs) were estimated using the exponentiated regression coefficient for Cox proportional hazards analyses implemented in R (survival 3.2-13 run in R, version 4.1.2). The impact of including an interaction term as the fourth parameter in the Cox proportional hazards model (TGF-β2×IFNGR2) was investigated to compare the independent effects of TGF-β2 and IFNGR2 in models with and without the interaction term. To visualize the survival proportion at any given time for combinations of high and low TGF-β2 mRNA expression groups in the context of high and low IFNGR2 mRNA expression groups from the interaction model, we plotted and calculated the shift in the baseline OS curve for the 45 pbDMG patients from the fitted hazard functions. In these comparisons, the median OS times for the TGFB2high versus TGFB2low groups of patients was compared in patients who expressed either high or low levels of IFNGR2. A significant interaction effect from the interaction model indicated differences in OS times for the TGFB2high versus TGFB2low groups of patients, depending on the level of IFNGR2.

Example 13. Downregulation of biomarkers for anti-tumor antigen-presenting cells (APCs) in pbDMG tumors.

The expressions of CD14, CD163, and ITGAX mRNAs exhibited significant decreases of 1.64-fold (p=0.037), 1.75-fold (p=0.019), and 3.33-fold (p<0.0001), respectively, in pbDMG tumors. The CD86 mRNA expression in pbDMG patients showed a non-significant 1.42-fold decrease compared with that in normal brainstem/pons tissue (p=0.14). The impact of high levels of TGF-β2 in combination with low levels of these markers for antigen-presenting cells in pbDMG patients was investigated. Patients with low levels of CD14 and CD163 expression in combination with high levels of TGF-β2 exhibited worse OS outcomes, and these makers were also expressed at lower levels in pbDMG tumors compared with those in normal brainstem/pons tissue. The median overall survival time for 14 patients in the TGFB2high/CD14low group was 7.5 months (95% CI: 5-12; number of events=14), which was significantly shorter (log-rank p-value=0.007) than the median survival time for the 31 remaining patients at 13 months (95% CI: 8-15; number of events=29), whereas the median OS time for 9 patients in the TGFB2high/CD163low group was 7 months (95% CI: 5-NA; number of events=9) as compared with the median OS time for the 36 remaining patients at 11 months (95% CI: 8-15; number of events=34; log-rank p-value=0.014).

The median OS time for 13 patients in the TGFB2high/CD86low group was 7 months (95% CI: 5-NA; number of events=13), which was significantly shorter than the overall survival time for the 32 remaining patients at 13 months (95% CI: 8-15; number of events=30) (log-rank p-value=0.001), and the median OS time for 9 patients in the TGFB2high/ITGAXlow group was 7 months (95% CI: 5-NA; number of events=9), which was shorter than but not statistically significant compared with the median overall survival time for the remaining 36 patients at 11 months (95% CI: 8-14; number of events=34) (p=0.885).

Example 14. Amplified expression of TGF-β2 compared with those of TGFB1 and TGFB3 mRNAs in pbDMG patients and normal pons tissue.

Applicants have discovered that the brainstem/pons tissues from pbDMG patients showed a selective upregulation of TGF-β2 and downregulations of TGFB1 and TGFB3 when compared with those of the normal pons tissue, with a significant 2.84-fold decrease, 1.51-fold increase, and 4.08-fold decrease in TGFB1, TGFB2, and TGFB3 mRNA expressions (p<0.0001, 0.002, and <0.0001, respectively). The profile of the TGFB ligands differed markedly in the pbDMG brain stem/pons and normal pons tissues, whereby in the pbDMG samples, the TGF-β2 mRNA expression was significantly higher than those of the TGFB1 (1.54-fold in-crease; p=4.1×10−4) and TGFB3 (2.25-fold increase; p<0.0001), suggesting the specific upregulation of the TGF-β2 isoform in the pbDMG tumor tissue. When comparing the mRNAs of TGFB1 and TGF-β2 in the normal pons tissue, it was found that TGF-β2 had a significantly lower expression, with a 2.78-fold decrease (p<0.0001). Similarly, when comparing the mRNA expressions of TGF-β2 and TGFB3, TGF-β2 showed a highly significant 2.74-fold decrease in mRNA levels (p<0.0001).

1. (canceled)

2. A method for treating or ameliorating the symptoms of cancer in subjects in need, the method comprising:
selecting the subjects who have (a) one or both of JAK1 and STAT1 reduced and (b) TGF-β2 and IFNGR2 elevated;
administering a composition comprising an agent for suppressing expression of TGF-β2; and
administering a composition comprising interferon-gamma.

3. (canceled)

4. The method of claim 2, wherein the cancer is brain or spinal cancer, glioma, glioblastoma, diffuse intrinsic pontine glioma (DIPG), diffuse midline glioma (DMG), diffuse hemispheric glioma, or leptomeningeal or brain metastasis, or wherein cells of the cancer exhibit somatic mutations comprising H2-K27M, H3-K27M, or H3-G34 genomic variants.

5. The method of claim 2, wherein the agent for suppressing expression of TGF-β2 and the interferon-gamma are administered concurrently, simultaneously, sequentially, or separately in time.

6. The method of claim 2, wherein the composition and agents are administered by infusion or injection.

7. The method of claim 2, wherein the one or both of JAK1 and STAT1 have an mRNA level reduced below a median of a control group of subjects having the same cancer, and TGF-β2 and IFNGR2 elevated above a median of a control group of subjects having the same cancer.

8. The method of claim 2, wherein the agent for suppressing expression of TGF-β2 is selected from Table 1 or Table 2, and chemically-modified variants thereof, LNA variants thereof, gapmer variants thereof, and any combination or pooling thereof.

9. The method of claim 2, wherein the agent for inhibiting or suppressing expression of TGF-β2 is
SEQ ID NO: 137
C*G*G*C*A*T*G*T*C*T*A*T*T*T*T*G*T*A
(OT-101)
or
SEQ ID NO: 1
CGGCATGTCTATTTTGTA.

10. The method of claim 2, wherein the interferon-gamma is human, recombinant interferon-gamma.

11. The method of claim 2, wherein the agent or composition comprises a carrier of sterile water for injection, saline, isotonic saline, or a combination thereof.

12. The method of claim 2, wherein the agent or composition is substantially free of excipients.

13. The method of claim 2, wherein the composition is stable for at least 14 days in carrier at 37° C.

14. The method of claim 2, wherein the subject upon the administration or use has a reduced TGF-β2 expression.

15. The method of claim 2, wherein the administration or use decreases mortality rate at month 6, 12, 18, 24, 30, or 36.

16. The method of claim 2, wherein the administration or use increases survival rate at month 6, 12, 18, 24, 30, or 36.

17. The method of claim 2, wherein the administration or use of the composition is combined with a standard of care treatment for cancer, wherein the standard of care treatment comprises chemotherapy or radiation therapy.

19. (canceled)


SEQUENCE LISTING (136 sequences)

SEQ ID NO: 1 - DNA, 18 bp, synthetic construct
  source 1..18: mol_type = other DNA
cggcatgtctattttgta

SEQ ID NO: 2 - DNA, 21 bp, synthetic construct
  source 1..21: mol_type = other DNA
taaagttcgtttagagaacag

SEQ ID NO: 3 - DNA, 15 bp, synthetic construct
  source 1..15: mol_type = other DNA
agccctgtatacgac

SEQ ID NO: 4 - DNA, 19 bp, synthetic construct
  source 1..19: mol_type = other DNA
gtaggtaaaaacctaatat

SEQ ID NO: 5 - DNA, 20 bp, synthetic construct
  source 1..20: mol_type = other DNA
cgtttagagaacagatctac

SEQ ID NO: 6 - DNA, 20 bp, synthetic construct
  source 1..20: mol_type = other DNA
cattgtagatgtcaaaagcc

SEQ ID NO: 7 - DNA, 21 bp, synthetic construct
  source 1..21: mol_type = other DNA
ctccctcatggtggcagttga

SEQ ID NO: 8 - DNA, 20 bp, synthetic construct
  source 1..20: mol_type = other DNA
gttcgtttagagaacagatc

SEQ ID NO: 9 - DNA, 18 bp, synthetic construct
  source 1..18: mol_type = other DNA
tttgttcctggatgactc

SEQ ID NO: 10 - DNA, 18 bp, synthetic construct
  source 1..18: mol_type = other DNA
gtttgttcctggatgact

SEQ ID NO: 11 - DNA, 18 bp, synthetic construct
  source 1..18: mol_type = other DNA
agtttgttcctggatgac

SEQ ID NO: 12 - DNA, 18 bp, synthetic construct
  source 1..18: mol_type = other DNA
cagtttgttcctggatga

SEQ ID NO: 13 - DNA, 18 bp, synthetic construct
  source 1..18: mol_type = other DNA
tcagtttgttcctggatg

SEQ ID NO: 14 - DNA, 18 bp, synthetic construct
  source 1..18: mol_type = other DNA
ctcagtttgttcctggat

SEQ ID NO: 15 - DNA, 18 bp, synthetic construct
  source 1..18: mol_type = other DNA
tgtgtgtgtgtgcgtgtg

SEQ ID NO: 16 - DNA, 18 bp, synthetic construct
  source 1..18: mol_type = other DNA
gtgtgtgtgtgtgcgtgt

SEQ ID NO: 17 - DNA, 18 bp, synthetic construct
  source 1..18: mol_type = other DNA
tgtgtgtgtgtgtgcgtg

SEQ ID NO: 18 - DNA, 18 bp, synthetic construct
  source 1..18: mol_type = other DNA
gtgtgtgtgtgtgtgcgt

SEQ ID NO: 19 - DNA, 18 bp, synthetic construct
  source 1..18: mol_type = other DNA
tgtgtgtgtgtgtgtgcg

SEQ ID NO: 20 - DNA, 18 bp, synthetic construct
  source 1..18: mol_type = other DNA
gtgtgtgtgtgtgtgtgc

SEQ ID NO: 21 - DNA, 18 bp, synthetic construct
  source 1..18: mol_type = other DNA
tgtgtgtgtgtgtgtgtg

SEQ ID NO: 22 - DNA, 18 bp, synthetic construct
  source 1..18: mol_type = other DNA
gtgtgtgtgtgtgtgtgt

SEQ ID NO: 23 - DNA, 18 bp, synthetic construct
  source 1..18: mol_type = other DNA
tgtgtgtgtgtgtgtgtg

SEQ ID NO: 24 - DNA, 18 bp, synthetic construct
  source 1..18: mol_type = other DNA
gtgtgtgtgtgtgtgtgt

SEQ ID NO: 25 - DNA, 18 bp, synthetic construct
  source 1..18: mol_type = other DNA
tgtgtgtgtgtgtgtgtg

SEQ ID NO: 26 - DNA, 18 bp, synthetic construct
  source 1..18: mol_type = other DNA
gtgtgtgtgtgtgtgtgt

SEQ ID NO: 27 - DNA, 18 bp, synthetic construct
  source 1..18: mol_type = other DNA
tgtgtgtgtgtgtgtgtg

SEQ ID NO: 28 - DNA, 18 bp, synthetic construct
  source 1..18: mol_type = other DNA
gtgtgtgtgtgtgtgtgt

SEQ ID NO: 29 - DNA, 18 bp, synthetic construct
  source 1..18: mol_type = other DNA
cgtgtgtgtgtgtgtgtg

SEQ ID NO: 30 - DNA, 18 bp, synthetic construct
  source 1..18: mol_type = other DNA
gcgtgtgtgtgtgtgtgt

SEQ ID NO: 31 - DNA, 18 bp, synthetic construct
  source 1..18: mol_type = other DNA
tgcgtgtgtgtgtgtgtg

SEQ ID NO: 32 - DNA, 18 bp, synthetic construct
  source 1..18: mol_type = other DNA
gtgcgtgtgtgtgtgtgt

SEQ ID NO: 33 - DNA, 18 bp, synthetic construct
  source 1..18: mol_type = other DNA
agtggcggatctgaactc

SEQ ID NO: 34 - DNA, 18 bp, synthetic construct
  source 1..18: mol_type = other DNA
gagtggcggatctgaact

SEQ ID NO: 35 - DNA, 18 bp, synthetic construct
  source 1..18: mol_type = other DNA
gagtgtgctgcaggtaga

SEQ ID NO: 36 - DNA, 18 bp, synthetic construct
  source 1..18: mol_type = other DNA
tcgagtgtgctgcaggta

SEQ ID NO: 37 - DNA, 18 bp, synthetic construct
  source 1..18: mol_type = other DNA
atcgagtgtgctgcaggt

SEQ ID NO: 38 - DNA, 18 bp, synthetic construct
  source 1..18: mol_type = other DNA
gtgctgttgtagatggaa

SEQ ID NO: 39 - DNA, 18 bp, synthetic construct
  source 1..18: mol_type = other DNA
ggtgctgttgtagatgga

SEQ ID NO: 40 - DNA, 18 bp, synthetic construct
  source 1..18: mol_type = other DNA
tggtgctgttgtagatgg

SEQ ID NO: 41 - DNA, 18 bp, synthetic construct
  source 1..18: mol_type = other DNA
ctggtgctgttgtagatg

SEQ ID NO: 42 - DNA, 18 bp, synthetic construct
  source 1..18: mol_type = other DNA
cctggtgctgttgtagat

SEQ ID NO: 43 - DNA, 18 bp, synthetic construct
  source 1..18: mol_type = other DNA
ccctggtgctgttgtaga

SEQ ID NO: 44 - DNA, 18 bp, synthetic construct
  source 1..18: mol_type = other DNA
tccctggtgctgttgtag

SEQ ID NO: 45 - DNA, 18 bp, synthetic construct
  source 1..18: mol_type = other DNA
gtccctggtgctgttgta

SEQ ID NO: 46 - DNA, 18 bp, synthetic construct
  source 1..18: mol_type = other DNA
agtccctggtgctgttgt

SEQ ID NO: 47 - DNA, 18 bp, synthetic construct
  source 1..18: mol_type = other DNA
ctgggttggagatgttaa

SEQ ID NO: 48 - DNA, 18 bp, synthetic construct
  source 1..18: mol_type = other DNA
gctgggttggagatgtta

SEQ ID NO: 49 - DNA, 18 bp, synthetic construct
  source 1..18: mol_type = other DNA
cgctgggttggagatgtt

SEQ ID NO: 50 - DNA, 18 bp, synthetic construct
  source 1..18: mol_type = other DNA
tagcgctgggttggagat

SEQ ID NO: 51 - DNA, 18 bp, synthetic construct
  source 1..18: mol_type = other DNA
atgtagcgctgggttgga

SEQ ID NO: 52 - DNA, 18 bp, synthetic construct
  source 1..18: mol_type = other DNA
agccattcgccttctgct

SEQ ID NO: 53 - DNA, 18 bp, synthetic construct
  source 1..18: mol_type = other DNA
gtctttatggtgaagcca

SEQ ID NO: 54 - DNA, 18 bp, synthetic construct
  source 1..18: mol_type = other DNA
tgtctttatggtgaagcc

SEQ ID NO: 55 - DNA, 18 bp, synthetic construct
  source 1..18: mol_type = other DNA
ctgtctttatggtgaagc

SEQ ID NO: 56 - DNA, 18 bp, synthetic construct
  source 1..18: mol_type = other DNA
cctgtctttatggtgaag

SEQ ID NO: 57 - DNA, 18 bp, synthetic construct
  source 1..18: mol_type = other DNA
gttcctgtctttatggtg

SEQ ID NO: 58 - DNA, 18 bp, synthetic construct
  source 1..18: mol_type = other DNA
ggttcctgtctttatggt

SEQ ID NO: 59 - DNA, 18 bp, synthetic construct
  source 1..18: mol_type = other DNA
aggttcctgtctttatgg

SEQ ID NO: 60 - DNA, 18 bp, synthetic construct
  source 1..18: mol_type = other DNA
caggttcctgtctttatg

SEQ ID NO: 61 - DNA, 18 bp, synthetic construct
  source 1..18: mol_type = other DNA
ccaggttcctgtctttat

SEQ ID NO: 62 - DNA, 18 bp, synthetic construct
  source 1..18: mol_type = other DNA
ggtcttcccactgttttt

SEQ ID NO: 63 - DNA, 18 bp, synthetic construct
  source 1..18: mol_type = other DNA
aggagatgtggggtcttc

SEQ ID NO: 64 - DNA, 18 bp, synthetic construct
  source 1..18: mol_type = other DNA
caggagatgtggggtctt

SEQ ID NO: 65 - DNA, 18 bp, synthetic construct
  source 1..18: mol_type = other DNA
ggttggtctgttgtgact

SEQ ID NO: 66 - DNA, 18 bp, synthetic construct
  source 1..18: mol_type = other DNA
cggttggtctgttgtgac

SEQ ID NO: 67 - DNA, 18 bp, synthetic construct
  source 1..18: mol_type = other DNA
ccggttggtctgttgtga

SEQ ID NO: 68 - DNA, 18 bp, synthetic construct
  source 1..18: mol_type = other DNA
gagaatggttagaggttc

SEQ ID NO: 69 - DNA, 18 bp, synthetic construct
  source 1..18: mol_type = other DNA
gtagagaatggttagagg

SEQ ID NO: 70 - DNA, 18 bp, synthetic construct
  source 1..18: mol_type = other DNA
ggtgttttgccaatgtag

SEQ ID NO: 71 - DNA, 18 bp, synthetic construct
  source 1..18: mol_type = other DNA
gggtgttttgccaatgta

SEQ ID NO: 72 - DNA, 18 bp, synthetic construct
  source 1..18: mol_type = other DNA
tgggtgttttgccaatgt

SEQ ID NO: 73 - DNA, 18 bp, synthetic construct
  source 1..18: mol_type = other DNA
catcatcgttgtcgtcgt

SEQ ID NO: 74 - DNA, 18 bp, synthetic construct
  source 1..18: mol_type = other DNA
gaacggtacgtacagcaa

SEQ ID NO: 75 - DNA, 18 bp, synthetic construct
  source 1..18: mol_type = other DNA
ggaacggtacgtacagca

SEQ ID NO: 76 - DNA, 18 bp, synthetic construct
  source 1..18: mol_type = other DNA
aggaacggtacgtacagc

SEQ ID NO: 77 - DNA, 18 bp, synthetic construct
  source 1..18: mol_type = other DNA
taggaacggtacgtacag

SEQ ID NO: 78 - DNA, 18 bp, synthetic construct
  source 1..18: mol_type = other DNA
ataggaacggtacgtaca

SEQ ID NO: 79 - DNA, 18 bp, synthetic construct
  source 1..18: mol_type = other DNA
gataggaacggtacgtac

SEQ ID NO: 80 - DNA, 18 bp, synthetic construct
  source 1..18: mol_type = other DNA
ggataggaacggtacgta

SEQ ID NO: 81 - DNA, 18 bp, synthetic construct
  source 1..18: mol_type = other DNA
gggataggaacggtacgt

SEQ ID NO: 82 - DNA, 18 bp, synthetic construct
  source 1..18: mol_type = other DNA
gggtgcctattgcatagc

SEQ ID NO: 83 - DNA, 18 bp, synthetic construct
  source 1..18: mol_type = other DNA
agggtgcctattgcatag

SEQ ID NO: 84 - DNA, 18 bp, synthetic construct
  source 1..18: mol_type = other DNA
aagggtgcctattgcata

SEQ ID NO: 85 - DNA, 18 bp, synthetic construct
  source 1..18: mol_type = other DNA
gaagggtgcctattgcat

SEQ ID NO: 86 - DNA, 18 bp, synthetic construct
  source 1..18: mol_type = other DNA
ggaagggtgcctattgca

SEQ ID NO: 87 - DNA, 18 bp, synthetic construct
  source 1..18: mol_type = other DNA
tgggaagggtgcctattg

SEQ ID NO: 88 - DNA, 18 bp, synthetic construct
  source 1..18: mol_type = other DNA
atgggaagggtgcctatt

SEQ ID NO: 89 - DNA, 18 bp, synthetic construct
  source 1..18: mol_type = other DNA
aatgggaagggtgcctat

SEQ ID NO: 90 - DNA, 18 bp, synthetic construct
  source 1..18: mol_type = other DNA
gaatgggaagggtgccta

SEQ ID NO: 91 - DNA, 18 bp, synthetic construct
  source 1..18: mol_type = other DNA
agaatgggaagggtgcct

SEQ ID NO: 92 - DNA, 18 bp, synthetic construct
  source 1..18: mol_type = other DNA
aagaatgggaagggtgcc

SEQ ID NO: 93 - DNA, 18 bp, synthetic construct
  source 1..18: mol_type = other DNA
taagaatgggaagggtgc

SEQ ID NO: 94 - DNA, 18 bp, synthetic construct
  source 1..18: mol_type = other DNA
gtaagaatgggaagggtg

SEQ ID NO: 95 - DNA, 18 bp, synthetic construct
  source 1..18: mol_type = other DNA
agtaagaatgggaagggt

SEQ ID NO: 96 - DNA, 18 bp, synthetic construct
  source 1..18: mol_type = other DNA
gagtaagaatgggaaggg

SEQ ID NO: 97 - DNA, 18 bp, synthetic construct
  source 1..18: mol_type = other DNA
cagactttctcggtcata

SEQ ID NO: 98 - DNA, 18 bp, synthetic construct
  source 1..18: mol_type = other DNA
gcagactttctcggtcat

SEQ ID NO: 99 - DNA, 18 bp, synthetic construct
  source 1..18: mol_type = other DNA
tgcagactttctcggtca

SEQ ID NO: 100 - DNA, 18 bp, synthetic construct
  source 1..18: mol_type = other DNA
atgcagactttctcggtc

SEQ ID NO: 101 - DNA, 18 bp, synthetic construct
  source 1..18: mol_type = other DNA
aatgcagactttctcggt

SEQ ID NO: 102 - DNA, 18 bp, synthetic construct
  source 1..18: mol_type = other DNA
taatgcagactttctcgg

SEQ ID NO: 103 - DNA, 18 bp, synthetic construct
  source 1..18: mol_type = other DNA
gacctggacttttttccc

SEQ ID NO: 104 - DNA, 18 bp, synthetic construct
  source 1..18: mol_type = other DNA
tgacctggacttttttcc

SEQ ID NO: 105 - DNA, 18 bp, synthetic construct
  source 1..18: mol_type = other DNA
ctgacctggacttttttc

SEQ ID NO: 106 - DNA, 18 bp, synthetic construct
  source 1..18: mol_type = other DNA
gctgacctggactttttt

SEQ ID NO: 107 - DNA, 18 bp, synthetic construct
  source 1..18: mol_type = other DNA
ctgcaatgatgtggcaaa

SEQ ID NO: 108 - DNA, 18 bp, synthetic construct
  source 1..18: mol_type = other DNA
tctgcaatgatgtggcaa

SEQ ID NO: 109 - DNA, 18 bp, synthetic construct
  source 1..18: mol_type = other DNA
ttctgcaatgatgtggca

SEQ ID NO: 110 - DNA, 18 bp, synthetic construct
  source 1..18: mol_type = other DNA
cttctgcaatgatgtggc

SEQ ID NO: 111 - DNA, 18 bp, synthetic construct
  source 1..18: mol_type = other DNA
gctgcccacttgcatact

SEQ ID NO: 112 - DNA, 18 bp, synthetic construct
  source 1..18: mol_type = other DNA
gttggcagaacatagaac

SEQ ID NO: 113 - DNA, 18 bp, synthetic construct
  source 1..18: mol_type = other DNA
cgttggcagaacatagaa

SEQ ID NO: 114 - DNA, 18 bp, synthetic construct
  source 1..18: mol_type = other DNA
gcgttggcagaacataga

SEQ ID NO: 115 - DNA, 18 bp, synthetic construct
  source 1..18: mol_type = other DNA
atggggctacaggggata

SEQ ID NO: 116 - DNA, 18 bp, synthetic construct
  source 1..18: mol_type = other DNA
tatggggctacaggggat

SEQ ID NO: 117 - DNA, 18 bp, synthetic construct
  source 1..18: mol_type = other DNA
ttatggggctacagggga

SEQ ID NO: 118 - DNA, 18 bp, synthetic construct
  source 1..18: mol_type = other DNA
agttatggggctacaggg

SEQ ID NO: 119 - DNA, 18 bp, synthetic construct
  source 1..18: mol_type = other DNA
aagttatggggctacagg

SEQ ID NO: 120 - DNA, 18 bp, synthetic construct
  source 1..18: mol_type = other DNA
caagttatggggctacag

SEQ ID NO: 121 - DNA, 18 bp, synthetic construct
  source 1..18: mol_type = other DNA
ccaagttatggggctaca

SEQ ID NO: 122 - DNA, 18 bp, synthetic construct
  source 1..18: mol_type = other DNA
tccaagttatggggctac

SEQ ID NO: 123 - DNA, 18 bp, synthetic construct
  source 1..18: mol_type = other DNA
atccaagttatggggcta

SEQ ID NO: 124 - DNA, 18 bp, synthetic construct
  source 1..18: mol_type = other DNA
tatccaagttatggggct

SEQ ID NO: 125 - DNA, 18 bp, synthetic construct
  source 1..18: mol_type = other DNA
ctatccaagttatggggc

SEQ ID NO: 126 - DNA, 18 bp, synthetic construct
  source 1..18: mol_type = other DNA
attggaggaaatagggtg

SEQ ID NO: 127 - DNA, 18 bp, synthetic construct
  source 1..18: mol_type = other DNA
gtcttgtaggtagcagcc

SEQ ID NO: 128 - DNA, 18 bp, synthetic construct
  source 1..18: mol_type = other DNA
ggtcttgtaggtagcagc

SEQ ID NO: 129 - DNA, 18 bp, synthetic construct
  source 1..18: mol_type = other DNA
tggtcttgtaggtagcag

SEQ ID NO: 130 - DNA, 18 bp, synthetic construct
  source 1..18: mol_type = other DNA
ctggtcttgtaggtagca

SEQ ID NO: 131 - DNA, 18 bp, synthetic construct
  source 1..18: mol_type = other DNA
tctggtcttgtaggtagc

SEQ ID NO: 132 - DNA, 18 bp, synthetic construct
  source 1..18: mol_type = other DNA
gtctggtcttgtaggtag

SEQ ID NO: 133 - DNA, 18 bp, synthetic construct
  source 1..18: mol_type = other DNA
agtctggtcttgtaggta

SEQ ID NO: 134 - DNA, 18 bp, synthetic construct
  source 1..18: mol_type = other DNA
gagtctggtcttgtaggt

SEQ ID NO: 135 - DNA, 18 bp, synthetic construct
  source 1..18: mol_type = other DNA
ggagtctggtcttgtagg

SEQ ID NO: 136 - DNA, 18 bp, synthetic construct
  source 1..18: mol_type = other DNA
aggagtctggtcttgtag